(19) United States
(12) Patent Application Publication
(10) Pub. No.: US 2021/0062263 A1
(43) Pub. Date: Mar. 4, 2021

(54) A CLINICAL MANAGEMENT PROTOCOL

(71) Applicant: Graeme SOUTHWICK, (US)

(72) Inventors: Peter TEMPLE-SMITH, Victoria (AU); Seungmin HAM, Victoria (AU); Graeme SOUTHWICK, Malvern (AU)

(21) Appl. No.: 16/644,594

(22) PCT Filed: Sep. 6, 2017
(86) PCT No.: PCT/AU2017/050964

§ 371 Date: Mar. 5, 2020

Related U.S. Application Data
(60) Provisional application No. 62/383,916, filed on Sep. 6, 2016.
(60) Provisional application No. 62/487,667, filed on Apr. 20, 2017.

(51) Int. Cl. C12Q 1/6883; A61K 38/17

(52) U.S. Cl. CPC C12Q 1/6883; A61K 45/06; A61K 38/1709

(57) ABSTRACT

The present invention relates generally to an assay for use in a clinical protocol to manage the extent of scarring or potential scarring associated with wound healing in human and animal subjects. The assay comprises an assessment of the likelihood of aberrant scar formation associated with fibrosis by screening for time-related sensitivity to an activin in fibroblasts. A treatment regime is proposed for subjects at risk of aberrant scar formation. The present invention is applicable to surface wounds and internal wounds.

This application is associated with and claims priority from U.S. Provisional Patent Application No. 62/373,916, filed on 6 Sep. 2016, entitled “A method of treatment and prophylaxis” AND U.S. Provisional Patent Application No. 62/487,667, filed on 20 Apr. 2017, entitled “A clinical management protocol”, the entire contents of which, are incorporated herein by reference, in their entirety.

### FIELD

The present invention relates generally to an assay for use in a clinical protocol to manage the extent of scarring or potential scarring associated with wound healing in human and animal subjects. The assay comprises an assessment of the likelihood of aberrant scar formation associated with fibrosis. A treatment regime is proposed for subjects at risk of aberrant scar formation. The present invention is applicable to surface wounds and internal wounds.

### BACKGROUND

Bibliographic details of the publications referred to by author in this specification are collected alphabetically at the end of the description.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavor to which this specification relates.

Wound healing is a complex, multifaceted process with intertwining temporal and spatial relationships and includes phases of inflammation, proliferation and remodeling (Guo et al. (2010) Journal Dent Res 89:219-229; Shih et al. (2010) Wound Repair Regeneration 18:139-153). Some aspects of wound healing can lead to aberrant conditions such as abnormalities in inflammation, cell migration and proliferation, angiogenesis, neovascularization, formation of granulation tissue and collagen deposition (Usui et al. (2008) Journal of Histochem Cytochem 56:687-696; Mustoe et al. (2004) Amer Journal Surgery 187:655-705). Fibrosis develops following a thickening of connective tissue, frequently following injury and during the wound healing process. Growth factors such as the activins and cytokines are generally implicated.

The activins are members of the transforming growth factor (TGF)-β superfamily. Whilst overexpression of activins can accelerate wound healing, this acceleration can lead to development of fibrosis at the wound site.

Keloids are a benign form of tumor caused by fibrosis during and after wound healing. Keloids are characterized by an over population of fibroblasts which deposit an excessive amount of components of the extracellular matrix (ECM) such as collagen, fibronectin, elastin and prostaglandins. A keloid or keloidal scar (Rapini et al. (2007) Dermatology: 2 volume set, St. Louis, Mosby at p 1499) can form at the site of a healed wound and is a result of overgrowth of granulation tissue, containing generally type III (early) collagen. Over time, the collagen is replaced by type I (late) collagen. Whilst a keloid scar is benign it can result in disfiguring and discomfort to the affected subject.

Treatment of keloids is complex and difficult and can be age dependent, causation dependent and ranges from preventative to interventionist including, laser therapy, corticosteroids, pressure therapy, surgery, radiotherapy or combinations of these (Amo et al. (2014) BUMS 40(7):1255-1266; Gauglitz et al. (2011) Molecular Medicine 17(1-2):113-125; Andrews et al. (2016) Matrix Biology 51:37-46).

There is a need to develop a procedure to predict if a subject is likely to exhibit aberrant scarring associated with fibrotic aspects of wound healing. Such a procedure enables a clinical management protocol regime to be instigated to minimize or mitigate aberrant scar formation or its potential to form.

### SUMMARY

The present invention teaches an assay for use in a clinical management protocol to reduce aberrant scar formation associated with wound healing. The wounds may be external (dermal) or internal. The assay may be referred to as a “scar predictability test” or a “keloid/hypertrophic scar therapeutic test” and assesses a subject's response or likely response to the wound healing process. The aim of the test is to recognize response patterns in human and animal subjects associated with a likely keloid or hypertrophic scar outcome. The test also allows assessment of an already formed scar and healing area around a wound so that a therapeutic program can be instigated to provide an improved predictable outcome. The scar predictability test is based on the level of sensitivity of dermal and other fibroblasts including internal fibroblasts to activin over time. Generally, a biopsy specimen comprising fibroblasts is employed in the assay. Fibroblast sensitivity to activin is a measure of the likely level of scarring or the propensity for an already formed scar to treatment. In an embodiment, a high sensitivity is indicative of a high likelihood of adverse fibrotic scar formation. Low sensitivity is an indicator of a lower likelihood of aberrant or excessive scar formation. By an “aberrant scar formation” includes the development of keloids and hypertrophic scarring. The assay applicable for external (dermal) and scarring as well as internal wounds and scarring such as around the bowel, urinary tract or other anatomical sites.

The scar predictability test is, therefore, in an embodiment, a keloid/hypertrophic scar therapeutic test. A subject who, based on the test, is likely to exhibit aberrant scar formation is treated with an activin inhibitor such as but not limited to a TGF-β antagonist or inhibitor of a member of the Activator protein-1 (AP-1) family of transcription factors. A “TGF-β antagonist” includes a TGF-β1, 2 and 3 antagonist. In an example, the TGF-β antagonist is follistatin, PB-01 (Paranta Biosciences Ltd, Victoria, Australia) or a functional variant or isoform thereof or an AP-1 inhibitor. In an embodiment, the AP-1 inhibitor inhibits any one or more of Jun (v-Jun, c-Jun, Jun-8 or JunD), Fos (v-Fos, c-Fos, FosB, Fra1 or Fra2), ATF (ATF2, ATF3/LRF1, B-ATF, JDP1 or JDP3), and/or MAF (c-MAF, MAFB, MAFA, MAFG/F/K or Ncl). Other useful antagonists include an inhibitor of cAMP response element binding (CREB) protein and an inhibitor of prostaglandin E2 (PGE2). Hence, the development of a scar predictability test enables development of a clinical management protocol to reduce the incidence or risk of aberrant scar formation, including keloids or hypertrophic scarring. The protocol can also be used for existing scars.

Accordingly, the present invention teaches the application of an activin inhibitor to treat fibrosis such as fibrotic conditions of the skin or sub-layers of the skin or internal tissue in subjects who have a scar predictability test result indicative of a high likelihood of aberrant scar development. The present invention extends to the application of the activin inhibitor to prevent aberrant scar development or to treat an existing aberrant scar. In an embodiment, the activin inhibitor is selected from the group consisting of a TGF-β antagonist and an AP-1 inhibitor. In an embodiment, the TGF-β antagonist is follistatin, PB-01 or a functional variant or isoform thereof or an AP-1 inhibitor. The treatment of fibrosis includes the treatment of inflammatory aspects associated with fibrosis such as those which pre-empt a fibrotic event.

Accordingly, taught herein is an assay to assess likely extent of scar formation at the site of a wound or potential wound in a subject, the method comprising contacting a sample of dermal fibroblasts or internal site fibroblasts from the subject with an activin and screening for time-related sensitivity to the activin; wherein high sensitivity compared to a control is indicative of a likelihood of aberrant scar development; wherein low sensitivity to the activin compared to a control is indicative of a likelihood of non-aberrant scar development. As indicated above, reference to “aberrant scar development” includes a fibrotic condition such as but not limited to keloids and/or hypertrophic scar formation. The sample, in an embodiment, includes a biopsy comprising dermal fibroblasts or internal site fibroblasts. An internal site includes the site of a wound or potential wound such as following a surgical procedure. The scar may be a potential scar or an existing scar. The level of sensitivity is based on gene, miRNA and/or protein expression profiles in response to activin or other indicator of activin-mediated signaling. In an embodiment, a response to different concentrations of activins is measured over time.

Where there is a likelihood of aberrant scar development, or where an aberrant scar has developed, a clinical management treatment protocol is implemented. The test provides patterns of recognition of aberrant scar formation. It is applicable for surface and internal wounds or potential wounds such as resulting from a surgical procedure including a biopsy. Enabled herein is a clinical management protocol to assess likely extent of aberrant scar formation at the site of a wound or potential wound in a subject, the method comprising contacting a sample of fibroblasts from the healing area from the subject with an activin and screening for time-related sensitivity to the activin wherein a rapid change in gene, miRNA and/or protein expression profile, or other indicator of activin-mediated signaling, in response to activin compared to a control is indicative of a likelihood of aberrant scar development; wherein a slow change in expression profile compared to a control is indicative of a likelihood of non-aberrant scar development.

Taught herein is a method for the treatment of fibrosis in a subject, the method comprising contacting a sample of dermal fibroblasts or internal site fibroblasts from the subject with an activin and screening for a level of sensitivity to the activin wherein a subject selected as exhibiting high sensitivity to activin compared to a control is administered an activin inhibitor for a time and under conditions sufficient to reduce the effects of fibrosis. Administration includes via topical application and injection or via any other convenient means. An ‘Injection” includes intravenous administration. Encompassed herein is, in an embodiment, parenteral administration. Further taught herein is the treatment of an inflammatory condition associated with fibrosis in a subject, the method comprising contacting a sample of dermal fibroblasts or internal site fibroblasts from the subject with an activin and screening for a level of sensitivity to the activin wherein a subject selected as exhibiting high sensitivity to the activin compared to a control is administered an activin inhibitor for a time and under conditions sufficient to reduce the effects of inflammation. In an embodiment, the fibrosis is associated with a wound or skin condition and the activin inhibitor is applied to or near the wound or skin condition. In an embodiment, the fibrosis is associated with an internal wound. In an embodiment, the activin inhibitor is a TGF-β antagonist, an AP-1 inhibitor, an inhibitor of cAMP response element binding (CREB) protein or an inhibitor of prostaglandin E2 (PGE2). As indicated above, a TGF-β antagonist includes a TGF-β1, 2 and 3 antagonist. In an example, the TFGβ antagonist is follistatin, PB-01 or a functional variant or isoform thereof or an AP-1 inhibitor. In an embodiment, the subject is a human although the present invention extends to the treatment of non-human animals. Hence, the present invention has human and veterinary applications. The fibrosis or inflammatory condition associated with fibrosis contemplated herein is selected from the group consisting of, but not limited to, fibrosis associated with surgical trauma or injury, Dupuytren's disease including Dupuytren's contracture, the site of a microbial or viral infection, an insect bite, pimples or other skin lesions including an ulcer, psoriasis, limited or diffuse scleroderma, eczema, a scratch mark, stretch marks (striae), acne, a burn, sunburn, a site of body piercing as well as melanomas and cancer scars such as skin cancer scars, as well as dermatomyositis or other autoimmune disease. Wounds and scarring internally such as around the bowel, urinary tract or an organ are also contemplated herein including intrajoint scars such as of the shoulder & upper limb including the wrist and hand, the lower limb including the ankle, knee or hip joints. A wound scar includes a keloid or hypertrophic scar.

In an embodiment, the wound or skin condition or fibrosis is exacerbated by a condition selected from the group consisting of type 1 or 2 diabetes, obesity, aging, coronary heart disease, peripheral vascular disease, wound or skin infection, cancer including melanoma, immunosuppression and the effects of radiation or chemotherapy as well as surgery or other trauma or dermatomyositis or other autoimmune disease. The present invention extends to the treatment of keloids and other fibrotic events in a subject whether or not of known etiology and any inflammatory events associated therewith wherein the treatment comprises selecting the subject based on the assay for the scar predictability test. A subject is selected for scar mitigation therapy where the subject's dermal fibroblasts or other internal fibroblasts are highly sensitive to the activin based on gene, miRNA and/or protein expression profiles or other indicator of activin-mediated signaling. The subject may have an existing scar or is likely to develop an aberrant scar after a procedure or natural healing.

A wound includes an external skin wound. In an embodiment, the wound is a skin wound or skin condition which affects one or more of the epidermal, dermal or subdermal layers such as the hypodermal layer. A wound may also be at an internal site such as wounding or scars about the bowel, urinary tract or an organ. Intrajoint scars such as of the shoulder & upper limb including the wrist and hand, the lower limb including the ankle, knee or hip joints can also be assessed. An internal or external would includes a potential wound such as may arise following a surgical procedure or biopsy.

In an embodiment, the fibrotic condition is keloids. However, the subject invention extends to other fibrotic events or inflammatory conditions associated with fibrosis and includes Dupuytren's disease, psoriasis, scleroderma, eczema. striae, acne, burns, sunburn, melanoma scars and hypertrophic scars as well as dermatomyositis or other autoimmune based diseases.

In an embodiment, the activin inhibitor is formulated in a topical gel, hydrogel or nano-channel system enabling penetration of a skin or epithelial layer. An injectable or other parenteral formulation may also be employed. All other suitable forms of administration are encompassed by the present invention. Without limiting the present invention to any one therapy or mode of action, the activin inhibitor is provided in an amount to inhibit the activity of an activin or a downstream signaling component such as connective tissue growth factor (CTGF). The present invention extends to the selection of a dose of activin inhibitor or the use of additional treatment protocols depending on the profile of gene, microRNA and/or protein expression or other indicator of activin-mediated signaling in response to exposure to the activin inhibitor or following the development of inflammation and/or the subsequent fibrotic condition.

In an embodiment, a composition is provided comprising an activin inhibitor in a medium which permits slow or sustained release of the inhibitor over time. For example, the slow or sustained release may be at or near the site of a wound or skin condition. Such media comprise, for example, a patch, bandage, gel, hydrogel, ointment, subcutaneous implant, a stent, impregnated sutures or a surgical implant. In an embodiment, the composition is in a form suitable for use by injection. In an embodiment, a treatment protocol of a wound (internal or external) or skin condition or a protocol resulting in a wound such as surgery or biopsy includes the step of applying an activin inhibitor. This may be, for example, in the form of a gel or ointment or as part of an impregnated bandage or via a topical or injectable formulation. The application of the activin inhibitor can also occur following an in vivo surgical procedure or following an arthroscopy or angioplasty or other form of catheterization.

In an embodiment, the activin inhibitor inhibits or reduces development of keloids in subjects deemed at risk of aberrant scar development following the scar predictability test.

A diagnostic kit comprising agents to monitor the clearance or activity of activin in a dermal fibroblast sample or other internal fibroblast sample is also contemplated herein. A therapeutic kit is also contemplated herein for use in conjunction with a scar predictability test.

A list of abbreviations used throughout the subject specification are provided in Table 1.

TABLE 1
Abbreviations

| ABBREVIATION | DESCRIPTION |
|---|---|
| AP-1 | Activator protein-1 |
| CRE | cAMP response element |
| CREB protein | cAMP response element binding protein |
| CTGF | Connective tissue growth factor |
| ECM | Extracellular matrix |
| ELISA | Enzyme linked immunosorbent assay |
| FST | Follistatin |
| FST288 | Follistatin isoform, 288 amino acids in length |
| FST315 | Follistatin isoform, 315 amino acids in length |
| IL-6 | Interleukin-6 |
| INHBA | Activin A |
| INHBB | Activin B |
| LDS formulation | A topical nano-channel system developed by |
| | Lyotropic Delivery Systems, Jerusalem, Israel |
| PB-01 | FST288 from Paranta Biosciences Ltd, |
| | Victoria, Australia |
| PGE2 | Prostaglandin E2 |
| qRT-PCR | Real time quantitative reverse transcription |
| | polymerase chain reaction |
| TGF-β | Transforming growth factor-β |
| TNF-α | Tumor necrosis factor-α |

### BRIEF DESCRIPTION OF THE FIGURES

FIG. 1 is a photographic representation of a section of human abdominal skin showing the positioning of the glued on Teflon rings, into which the three nano-channel liquid formulations (LPA, LPB, LPC) [Lyotropic Delivery Systems, Jerusalem, Israel] and a gel formulation (LPD) were placed for 24 hour exposure to the skin surface, and the blue dye tattoos outlining the position of each ring after removal at the end of the exposure time. Teflon ring diameter—10 mm.

FIG. 2 is a graphical representation showing transcutaneous penetration of FST288 into human abdominal skin after 24 hour exposure of the skin surface to three nano-channel liquid formulations (LPA, LPB, LPC) [Lyotropic Delivery Systems, Jerusalem, Israel] and a gel formulation (LPD). Saline was used for the untreated control and unloaded nano-channel liquid formulation was used as the vehicle control. Each protein extract from the skin after exposure to the control formulation (saline only) and the nano-channel liquid and gel formulations was assayed in triplicate to produce a technical mean and SEM. The FST288 content of each skin layer extract was normalized against the total protein content of the extract to provide a relative presence of FST288 in the extract against skin exposed to the control formulations.

FIG. 3 is a graphical representation showing transcutaneous penetration of FST288 into human eyelid skin after a single 24 hour exposure of the skin surface to one nano-channel liquid formulation (LPA) and a gel formulation (LPD). Unloaded nano-channel liquid formulation was used as the vehicle control (con). Each protein extract was assayed in triplicate to produce a technical mean and SEM. The FST288 content of each skin layer extract was normalized against the total protein content of the extract to provide a relative presence of FST288 in the extract.

FIGS. 4A through C are graphical representations showing that testosterone treatment delays wound healing. Comparison of A. wound area (cm²); B. wound width (μm); and C. epidermal thickness (μm) measured at days 3, 5, 7 and 14 post-wounding in intact and castrated males with or without testosterone implants. Wound closure is faster in testosterone-deprived mice. Intact: intact plus vehicle; Intact+T: intact plus testosterone; Castrated: castrated plus vehicle; Castrated+T: castrated plus testosterone. Data expressed as mean±SEM. N=6 per group. Means marked with different letters are significantly different (p<0.05).

FIGS. 5A through D are graphical representations showing testosterone stimulated increased activin A, follistatin, interleukin-6 (IL-6) and tumor necrosis factor-α factor-α (TNF-α) at wound sites in male mice. Effects of castration and testosterone replacement in male mice on skin levels of A. activin A, B. follistatin, C. IL-6 and D. TNF-α following injury (n=6 per group). Intact: intact males treated with vehicle; Intact+T: intact males treated with testosterone; Castrated: castrated males treated with vehicle; Castrated+T: castrated males treated with testosterone. Data expressed as mean±SEM; n=6 per group. Means marked with different letters are significantly different (p<0.05).

FIG. 6 is a graphical representation showing the role of follistatin in the treatment of Dupuytren's disease.

FIG. 7 is a schematic representation of the differential gene expression using RNA sequencing Heatmap (a) demonstrates upregulation and downregulation of selected genes which are against average of both normal and keloid fibroblasts at day 5 with/without 100 ng/ml follistatin treatment. The list of selected genes shows False Discovery Rate (FDR) with P values and Absolute log Fold-Change (Abs log FC) with upregulation and downregulation (b).

FIG. 8 is a graphical representation showing the effects of activin A in human dermal fibroblasts from normal and keloid tissues relative gene expression was measured by qRT-PCR with/without 200 pM activin A treatment for 24 hours. Basal keloid fibroblasts have significantly higher INHBA (a) and IL-6 (b) gene expression than normal controls. After activin A treatment, INHBA, CTGF (b), IL-6, PAI1 (e), FOSB (g), JUNB (h), and TGFB2 (m) gene expression in both normal and keloid fibroblasts was significantly upregulated compared to untreated fibroblasts using a single patient. CTGF expression was increased in activin treated fibroblasts from multiple patients (n). [vc; vehicle control and ACT; activin A 200 pM].

### DETAILED DESCRIPTION

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method steps or group of elements or integers or method steps.

As used in the subject specification, the singular forms “a”, “an” and “the” include plural aspects unless the context clearly dictates otherwise. Thus, for example, reference to “a fibrotic condition” includes a single fibrotic condition, as well as two or more fibrotic conditions; reference to “an agent” includes a single agent, as well as two or more agents; reference to “the disclosure” includes a single and multiple aspects taught by the disclosure; and so forth. Aspects taught and enabled herein are encompassed by the term “invention”. Any variants and derivatives contemplated herein are encompassed by “forms” of the invention.

Taught herein is an assay to assess the likelihood or otherwise of a subject developing or having developed an aberrant scarring associated with wound healing. The present assay is predicated in part on response pattern recognition in a subject based on likely extent of keloid or hypertrophic scar outcome. Aberrant scarring results from fibrosis and conditions such as keloids and hypertrophic scarring. The area affected may be an existing scar or healing area or the site of a potential wound such as following a surgical procedure or biopsy or condition. Accordingly, the assay comprises contacting a sample of dermal fibroblasts or internal site fibroblasts from the subject with an activin and screening for a level of sensitivity by the dermal fibroblast cells to the activin. Sensitivity is based on the profile of gene, miRNA and/or protein expression or other indicator of activin-mediated signaling, over time in response to different concentrations of activin. A gene, miRNA and/or protein expression profile (or other indicator of activin-mediated signaling) associated with high sensitivity to activin is indicative of a potential for aberrant scar formation. Low sensitivity is indicative of a lower likelihood of aberrant scar formation. For subjects where aberrant scarring is likely, i.e. dermal or internal fibroblasts highly sensitive to activin, a therapeutic management protocol is implemented. This is also the case for an existing scar where the subject has highly sensitive fibroblasts. Enabled herein is a method for preventing or treating a fibrotic condition or an inflammatory condition associated with a fibrotic condition is contemplated in subjects which are deemed to be at risk of aberrant scarring, the method comprising administering to a subject of an activin inhibitor. Administration includes topical and injection administration or any other suitable means of application of the inhibitor such as via parenteral administration. In an embodiment, the activin inhibitor is a TGF-β antagonist and/or an AP-1 inhibitor. A “TGF-β antagonist” includes any one or more of a TGF-β1, 2 or 3 antagonist. It is noted that TGF-β2 is regulated by activin A An example includes follistatin, PB-01 or a functional variant or isoform thereof or an AP-1 inhibitor. In an embodiment, the AP-1 inhibitor inhibits any one or more of Jun (v-Jun, c-Jun, Jun-8 or JunD), Fos (v-Fos, c-Fos, FosB, Fra1 or Fra2), ATF (ATF2, ATF3/LRF1, B-ATF, JDP1 or JDP3), and/or MAF (c-MAF, MAFB, MAFA, MAFG/F/K or Ncl). Other antagonists contemplated for use herein includes an inhibitor of cAMP response element binding (CREB) protein and an inhibitor prostaglandin E2 (PGE2). In an embodiment, the fibrotic condition or its associated inflammatory condition is of the skin or its layers, including the epidermal, dermal and hypodermal layers. In an embodiment, the fibrotic condition is in or at an internal tissue such as around an organ or tract such as around the bowel or urinogenital tract. The area affected may be an existing scar or site of a potential scar. The fibrotic condition which is usually preceded by an inflammatory response includes but is not limited to keloids leading to keloidal scarring at the site of a superficial wound of the skin or its layers or at a wound inside the body (internal wound). The fibrotic condition may be or arise from surgery, trauma, Dupuytren's disease, microbial or viral infection, insect bites, pimples or other skin lesions including ulcers, psoriasis, limited or diffuse scleroderma, eczema. scratching, stretch marks (striae), acne, burns, sunburn and body piercing as well as melanomas and cancer scars such as skin cancer scars or dermatomyositis or other autoimmune disease. A wound also includes a hypertrophic scar whether or not in a keloid state. The site of the fibrosis such as the keloids includes any site of trauma and includes the central chest region, back and shoulders including collar bone region, neck, head including the face and nose, ears, ear lobes, upper limbs (upper arms and lower arms including elbows, wrists, hands, fingers and thumbs), lower limbs (thighs, knees, legs, ankles, feet and toes), and pelvic region. Internal sites include areas around the bowel or urinogenital tract or any other anatomical site. The present invention is predicated in part on the surprising determination that time-related activin sensitivity provides an indicator of the likelihood of aberrant scar formation. The faster or more extensive the change in gene, miRNA and/or protein expression profile, or change in other indicators of activin-mediated signaling, the more likely an aberrant scar will form. The slower the response to activin is indicative of a lesser likelihood of aberrant scar formation. An aberrant scar comprises keloid or hypertrophic scarring. In high scar risk subjects, an activin inhibitor is administered to an external skin surface or to an internal anatomical site to reduce the incidence of aberrant fibroblast activity and reduces or ameliorates the formation of fibrosis such as keloids, hypertrophic scars and other collagen deposition type conditions as well as inflammatory conditions associated with fibrosis. Administration may be by any means suitable to the condition being treated including by any form of parenteral administration. Examples include topical and injectable administration.

The fibrosis may also result from or be exacerbated by a condition which is associated with delayed wound healing such as but not limited to resulting from type 1 or 2 diabetes, ulcers, obesity, increasing age of a subject, coronary heart disease, peripheral vascular disease, wound or skin infection, cancer including melanoma and immunosuppression and the effects of radiation or chemotherapy or dermatomyositis or other autoimmune disease. The present invention extends to the treatment of keloids, hypertrophic scars and other fibrotic events whether or not of known etiology and any inflammatory events associated therewith. Such a treatment protocol is nevertheless subject to the results of the scar predictability test.

The determination of the gene, miRNA and/or protein concentrations or levels or of other indicators of activin-mediated signaling enables establishment of a diagnostic rule based on the expression profile relative to a control. Alternatively, the diagnostic rule is based on the application of a statistical and machine learning algorithm. Such an algorithm uses relationships between the indicators and activin sensitivity status observed in training data (with known level of sensitivity) to infer relationships which are then used to predict the status of subjects with unknown status in relation to activin sensitivity. An algorithm may be employed which provides an index of probability that a subject has high or low sensitivity to activin.

Hence, the present invention contemplates the use of a knowledge base of training data comprising levels of indicators from dermal fibroblasts or internal site fibroblasts derived from a subject with known activin sensitivity status to generate a baseline from which a second knowledge base of data comprising levels of the same indicators from a subject with an unknown activin sensitivity status is compared to provide an index of probability that predicts the level of sensitivity to activin.

The term “training data” includes knowledge of levels of indicators relative to a control. A “control” includes a comparison to levels of indicators in a subject of known activin sensitivity status or may be a statistically determined level based on trials. The term “levels” also encompasses ratios of levels of indicators.

The “training data” also include the concentration of one or more of the indicators of activin-mediated signaling such as levels of gene, miRNA and/or protein expression. A clinical management protocol is then implemented in subjects with a high risk of aberrant scar development.

Accordingly, enabled herein is a method for the prevention or treatment of a fibrotic or an inflammatory condition associated therewith in a subject, the method comprising contacting a sample of dermal fibroblasts or internal site fibroblasts from the subject with an activin and screening for time-related sensitivity to the activin wherein a rapid change in gene, miRNA and/or protein expression profile, or other indicator of activin-mediated signaling, in response to activin compared to a control is indicative of a likelihood of aberrant scar development in a subject in need of treatment administering to the subject in need of treatment, an amount of an activin inhibitor effective to ameliorate the fibrotic or inflammatory condition. Administration may be by any means including parenteral means such as via topical or injection administration. Taught herein is a clinical management protocol to assess likely extent of aberrant scar formation at the site of a wound or potential wound in a subject, the method comprising contacting a sample of fibroblasts from the healing area from the subject with an activin and screening for time-related sensitivity to the activin wherein a rapid change in gene, miRNA and/or protein expression profile, or other indicator of activin-mediated signaling, in response to activin compared to a control is indicative of a likelihood of aberrant scar development; wherein a slow change in expression profile compared to a control is indicative of a likelihood of non-aberrant scar development.

In an embodiment, taught herein is a method for the prevention or treatment of a fibrotic condition or an inflammatory condition associated therewith of the skin or its layers in a subject, the method comprising contacting a sample of dermal fibroblasts or internal site fibroblasts from the subject with an activin and screening for time-related sensitivity to the activin wherein a rapid change in gene, miRNA and/or protein expression profile, or other indicator of activin-mediated signaling, in response to activin compared to a control is indicative of a likelihood of aberrant scar development; administering to the subject in need of treatment an amount of an activin inhibitor effective to ameliorate the fibrotic or inflammatory condition.

Also enabled herein is a method for the prevention or treatment of a fibrotic condition or an inflammatory condition associated therewith in a subject, the method comprising contacting a sample of dermal fibroblasts or internal site fibroblasts from the subject with an activin and screening for time-related sensitivity to the activin wherein a rapid change in gene, miRNA and/or protein expression profile, or other indicator of activin-mediated signaling, in response to activin compared to a control is indicative of a likelihood of aberrant scar development; administering to the subject in need of treatment an amount of an activin inhibitor effective to inhibit or otherwise suppress the activity of an activin and/or a downstream modulator.

In an embodiment, taught herein is a method for the prevention or treatment of a fibrotic condition or an inflammatory condition associated therewith of the skin or its layers in a subject, the method comprising contacting a sample of dermal fibroblasts or internal site fibroblasts from the subject with an activin and screening for time-related sensitivity to the activin wherein a rapid change in gene, miRNA and/or protein expression profile, or other indicator of activin-mediated signaling, in response to activin compared to a control is indicative of a likelihood of aberrant scar development; administering to the subject in need of treatment an activin inhibitor an amount of follistatin, PB-01 or a functional variant or isoform thereof effective to inhibit or otherwise suppress the activity of an activin and/or a downstream modulator.

As indicated above, administration may be by any convenient means including parenteral administration such as topical administration or by injection. The treatment of an epithelial wound includes a burn injury to such a surface. Wounds and skin conditions include fibrotic events and associated inflammatory conditions associated with surgical trauma or injury, Dupuytren's disease such as Dupuytren's contracture, the site of microbial or viral infection, an insect bite, pimples or other skin lesions including an ulcer, psoriasis, limited or diffuse scleroderma, eczema, a scratch mark, stretch mark (striae), acne, a burn, sunburn, a site of body piercing, melanomas and cancer scars such as skin cancer scars or following catheterization (e.g. arthroscopy or angioplasty) or dermatomyositis or other autoimmune disease.

The topical administration of an activin inhibitor to treat a wound means to topical administration at or near that particular wound or site of skin condition. Injectable administration is also contemplated herein. All forms of administration are encompassed by the present invention. Reference to ameliorating the fibrotic condition includes reducing the extent to which fibroblasts secrete excessive amounts of extracellular matrix (ECM) compounds such as collagen. The amelioration may also result from a reduction in the number of fibroblasts or active fibroblasts. The amelioration further includes reducing the extent to which the fibrotic condition forms or reduces its continued development if already formed. In another embodiment an inflammatory condition or event associate with fibrosis is ameliorated. In an embodiment, this effect by inhibition of an activin and/or a downstream modulator such as connective tissue growth factor (CTGF). By “topically administering” includes transcutaneous, subcutaneous, transdermal, transepithelial and subepithelial administration and the like. The treatment may be on or near a surface or subsurface skin wound or on an internal epithelial surface or layer. In an embodiment, administration is via a parenteral route.

Reference to an “activin” means activin A or activin B or activin AB. In an embodiment, the activin is activin A. All forms of activin A and B are encompassed by the present invention. Activin A is a dimeric protein comprising two activin PA subunits. Reference to “activin A” includes its natural variants and isoforms as well as its precursor, proprotein and intermediate forms. TGF-β2, for example, is regulated by activin A. Furthermore, the activin A promoter has a cAMP response element (CRE) site and prostaglandin E2 (PGE2) can increase the level of cAMP response element binding (CREB) protein). Activin B is a dimer protein comprising two β_{B} subunits. Reference to “activin B” includes its natural variants and isoforms as well as its precursor, proprotein and intermediate forms. Furthermore, the activin may be activin AB comprising β_{A} and β_{B} chains and its precursor, proprotein and intermediate forms.

In an embodiment, the fibrotic condition is keloids which includes a keloid scar. The subject method ameliorates the keloid meaning it reduces the extent to which it forms or reduces its continued development if already formed.

Hence, taught herein is a method for the prevention or treatment of a keloid condition in a subject, the method comprising contacting a sample of dermal fibroblasts or internal site fibroblasts from the subject with an activin and screening for time-related sensitivity to the activin wherein a rapid change in gene, miRNA and/or protein expression profile, or other indicator of activin-mediated signaling, in response to activin compared to a control is indicative of a likelihood of aberrant scar development; administering to the subject in need of treatment an amount of an activin inhibitor or a functional variant or isoform thereof effective to ameliorate the keloids.

In an embodiment, enabled herein is a method for the prevention or treatment of a keloid condition in a subject, the method comprising contacting a sample of dermal fibroblasts or internal site fibroblasts from the subject with an activin and screening for time-related sensitivity to the activin wherein a rapid change in gene, miRNA and/or protein expression profile, or other indicator of activin-mediated signaling, in response to activin compared to a control is indicative of a likelihood of aberrant scar development; administering to the subject in need of treatment an amount of an activin inhibitor effective to inhibit or otherwise suppress the activity of an activin and/or a downstream modulator.

As indicated above, reference to an activin means activin A or activin B or activin AB or various natural variants or isoforms thereof. A downstream modulator includes but is not limited to CTGF. Reference to an “activin inhibitor” includes inter alia follistatin, PB-01, or a functional variant or isoform thereof, a TGF-β antagonist (including any one of a TGF-β1, 2 or 3 antagonist) and an AP-1 inhibitor as well as other activin inhibitors such as an antibody. PB-01 is a TGF-β antagonist (Paranta Biosciences Ltd, Victoria, Australia). Other antagonists include an inhibitor of CREB protein and an inhibitor of PGE2.

Reference to a subject being treated includes humans and non-human primates, as well as a cow, horse, sheep, pig, goat, alpaca, llama, camel, dog or cat as well as a laboratory test animal such as a mouse, rat, guinea pig, hamster or rabbit. As indicated above, the fibrotic or associated inflammatory condition to be treated includes wounds and other trauma or conditions arising from or comprising injury, surgery, Dupuytren's disease, microbial or viral infection, an insect bite, pimples or other skin lesions including ulcers, psoriasis, scleroderma (limited or diffuse), eczema, hypertrophic scars, scratch marks, stretch marks (striae), acne, burns, sunburn, sites of body piercing as well as melanomas and cancer scars such as skin cancer scars and dermatomyositis or other autoimmune diseases. In addition, the wound or fibrotic or associated inflammatory condition may arise from or be exacerbated by type 1 or 2 diabetes, ulceration, obesity, age of a subject, coronary heart disease, peripheral vascular disease, wound or skin infection, cancer including melanoma and immunosuppression and effects of radiation or chemotherapy or dermatomyositis or other autoimmune disease. In an embodiment, a treatment protocol of a wound or skin condition or a protocol resulting in a wound such as surgery or biopsy includes the step of contacting a sample of dermal fibroblasts or internal site fibrobalsts from the subject with an activin and screening for time-related sensitivity to the activin wherein a rapid change in gene, miRNA and/or protein expression profile, or other indicator of activin-mediated signaling, in response to activin compared to a control is indicative of a likelihood of aberrant scar development and then, in subjects in need of treatment based on a high sensitivity to activin, applying the activin inhibitor. This may be, for example, in the form of a gel or ointment or as part of an impregnated bandage or an injectable. The application of an activin inhibitor can also occur following a surgical procedure or following an arthroscopy or angioplasty or other form of catheterization.

Enabled herein is a method for the treatment of a wound or skin condition in or on a subject in need of treatment, the method comprising contacting a sample of dermal fibroblasts or internal site fibroblasts from the subject with an activin and screening for time-related sensitivity to the activin wherein a rapid change in gene, miRNA and/or protein expression profile, or other indicator of activin-mediated signaling, in response to activin compared to a control is indicative of a likelihood of aberrant scar development and then administering to the wound or site of the skin condition and/or its surrounding region follistatin or PB-01 or a functional variant or isoform thereof for a time and under conditions sufficient to reduce the effects of fibrosis of the wound or site of the skin condition. This method also applies to existing scars. Other useful inhibitors include an inhibitor of CREB protein or an inhibitor of PGE2.

The follistatin used is generally from the same species of mammal as the subject being treated. The follistatin is then said to be homologous to the subject being treated. Hence, for example, human follistatin is used in humans, bovine follistatin is used in cows and so on. Notwithstanding, a heterologous mammalian follistatin can be used in a different mammal wherein the follistatin has been de-immunized or used in conjunction with an immunosuppressive agent. There is significant homology between some forms of mammalian follistatins hence in selected circumstances, a heterologous follistatin may be employed.

Any isoform or natural or artificially manufactured form (i.e. variant) of follistatin may be used. Reference can conveniently be made to International Patent Application No. PCT/AU2004/001253 and International Patent Application No. PCT/AU2004/001359. Reference to “follistatin” includes its preforms, pre-proforma, pre-secreted forms as well as any functional natural variant or isoform or functional artificially created derivative of follistatin.

“Variants” of follistatin include fragments, parts, portions or derivatives from either natural or non-natural sources and include isoforms. Non-natural sources include, for example, recombinant or synthetic sources. By “recombinant sources” is meant that the cellular source from which the follistatin is harvested has been genetically altered. This may occur, for example, in order to increase or otherwise enhance the rate and volume of production by that particular cellular source. Parts or fragments include, for example, active regions of follistatin. Variants may be derived from insertion, deletion or substitution of amino acids.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

Derivatives of nucleic acid sequences which may be utilized to express modified follistatin molecules may similarly be derived from single or multiple nucleotide substitutions, deletions and/or additions including fusion with other nucleic acid molecules.

A “variant” or “mutant” of the follistatin isoform should be understood to mean molecules which exhibit at least some of the functional activity of the form of follistatin of which it is a variant or mutant. A variation or mutation may take any form and may be naturally or non-naturally occurring.

A “homolog” is meant that the molecule is derived from a species other than that which is being treated in accordance with the method of the present invention. This may occur, for example, where it is determined that a species other than that which is being treated produces a form of follistatin isoform which exhibits similar and suitable functional characteristics to that of the follistatin isoform which is naturally produced by the subject undergoing treatment. Such derivatives and variants and isoforms also applies to PB-01.

In accordance with the present invention, it is determined that an activin inhibitor can be topically administered to the skin or internal layer of a subject deemed to be at risk of aberrant scar formation to thereby reduce the potential for a fibrotic condition such as keloids from developing or to reduce their further development once formed. The treatment can also ameliorate the effects of an inflammatory condition associated with a fibrotic condition. The activin inhibitor is therefore formulated in a manner to facilitate penetration of the activin inhibitor into at least the epidermal layer, optionally into the dermal layer and further optionally into the hypodermal layer. Hence, the topical formulation comprises an activin inhibitor and a medium which permits penetration through the skin to the site of the fibrotic condition or through an epithelial layer if the site of treatment is inside the body of the subject. The present invention extends to any parenteral formulation such as one suitable for injection. Conditions include but is not limited to the development of keloids or collagen-associated conditions around or with a surgical or trauma wound, the site of a Dupuytren's disease such as Dupuytren's contracture, site of a local microbial or viral infection, an insect bite, a pimple or other skin lesion, areas affected by psoriasis or limited or diffuse scleroderma, eczema, hypertrophic scars, a scratch, stretch mark (striae), acne, burn, sunburn, site of body piercing, melanomas and cancer scars such as skin cancer scars or dermatomyositis or other autoimmune disease. The activin inhibitor may also be a component in another treatment regime such as for type 1 or 2 diabetes, skin ulceration, obesity, age-related disorders, coronary heart disease, peripheral vascular disease, wound infection, cancer or immunosuppression. In addition, other active agents may be included such as a local anti-testosterone or other anti-androgen compound, an anti-microbial or anti-viral agent, an antibiotic, insulin or an anesthetic. Alternatively, the activin inhibitor may be used in combination with an estrogen to improve healing with reduced scar formation.

Wounds which can be effectively treated in accordance with the present invention include epidermal wounds involving cells and tissue in the epidermis (such as any of the five epidermal layers: stratum basale, stratum spinosum, stratum granulosum, stratum licidum, and stratum corneum); dermal wounds involving cells and tissue in the two layers of the dermis of the skin; and internal wounds at a particular anatomical site (e.g. an organ or tract). Thus, the methods and compositions of the present invention can be used to treat surface wounds such as skin abrasions, wounds involving injury to the dermis and epidermis, and also subsurface wounds such as enhancing closure of incisions following a surgical procedure. An internal wound or scar may also be treated. A wound may also be a hypertrophic scar. These are slow healing scars that are not necessarily keloid but can become so. They are red raised, limited to a site of injury and show long delay in healing to mature scar. The present invention further extends to the treatment of fibrosis or an inflammatory condition associated therewith in a subject by the topical administration or injection of an activin inhibitor or a functional variant or isoforms thereof in a subject deemed to be at risk of aberrant scar development following a scar predictability test.

By “treating wounds” or “treating a skin condition” it is meant promoting, accelerating and/or enhancing wound closure, wound contraction, maturation and remodeling, fibroplasia and granulation tissue formation, and/or re-epithelialization. In addition, “treating fibrosis” means the topical administration or injection of an activin inhibitor or its functional forms to treat fibrosis. Treating fibrosis also includes treating an inflammatory component which often pre-empts fibrosis. Treatment may be to prevent aberrant scar formation or to treat an existing scar.

One formulation medium comprises nano-sized, self-assembled liquid droplets which are capable of solubilizing the activin inhibitor. Alternatively, the medium comprises a modified lyotropic liquid crystalline structure of low viscosity, weak gel properties and high loading capability for the activin inhibitor. Such media are developed by, for example, and are available from Lyotropic Delivery Systems, Jerusalem, Israel. Both these media consist of water and oil nano-droplets, or nano-channels, which are thermodynamically stable. Other penetration enhancing formulations may also be employed such as surfactants, fatty acids, bile salts, chelating agents and non-chelating and non-surfactant agents. Reference can conveniently be made to U.S. Pat. No. 6,287,860.

Other topical media or an injectable may also be employed to facilitate penetration of the outer and inner skin and epithelial layers and include lotions, creams, gels, drops, suppositories, sprays, liquids, powders and ointments. The activin inhibitor formulation may also be part of a slow or sustained release formulation on any impregnated bandage, patch, stent, subcutaneous implant or impregnated slow or sustained release sutures. The activin inhibitor may also be associated with a catheter or instrument employed to take a biopsy. The topical or injectable medium comprising the activin inhibitor may comprise other active agents such as a local anti-testosterone or other androgen agent, an anti-microbial or anti-viral agent, an antibiotic, insulin or an anesthetic. In addition, the activin inhibitor may be used in combination with an estrogen. Any and all forms of parenteral formulations are contemplated for use herein.

Accordingly, enabled herein is a parenteral formulation comprising follistatin or a function variant or isoform thereof and one or more pharmaceutically acceptable carriers, diluents and/or excipients for use in a subject deemed to be at risk of aberrant scar formation following the scar predictability test. The formulation may alternatively comprise any of PB-01, an inhibitor of CREB protein and/or an inhibitor of PGE2. In an embodiment, the formulation is a topical or injectable formulation.

Further enabled is the use of an activin inhibitor in the manufacture of a medicament for the treatment of a fibrotic or associated inflammatory condition of the skin or within a skin layer for use in a subject deemed to be at risk of aberrant scar formation following the scar predictability test.

Still further taught is an activin inhibitor or a functional variant or isoform thereof for use in the treatment of a fibrotic for use in a subject deemed to be at risk of aberrant scar formation following the scar predictability test.

In an embodiment, the fibrotic condition is keloids.

Hence, enabled herein is the use of an activin inhibitor in the manufacture of a medicament for the treatment of keloids for use in a subject deemed to be at risk of aberrant scar formation following the scar predictability test.

In an embodiment, the activin inhibitor inhibits activin A or activin B or a downstream modulator such as CTGF.

In an embodiment, the fibrotic condition arises from or is exacerbated by Dupuytren's disease, the site of a microbial or viral infection, an insect bite, pimples or other skin lesions including an ulcer, psoriasis, limited or diffuse scleroderma, eczema, a hypertrophic scar or dermatomyositis or other autoimmune diseases. Hence, enabled herein is the use of an activin inhibitor in the manufacture of a medicament for the treatment of Dupuytren's disease, psoriasis, a form of scleroderma, dermatomyositis or other autoimmune diseases, eczema, a hypertrophic scar, a scratch mark, stretch mark (striae), a burn, sunburn, a site of body piercing, a melanoma, cancer scar, skin cancer scar, site of biopsy, site of a catheterization event or a surgical trauma or accidental trauma for use in a subject deemed to be at risk of aberrant scar formation following the scar predictability test. In an embodiment, the Dupuytren's disease is Dupuytren's contracture.

Hence, enabled herein is the use of an activin inhibitor in the manufacture of a medicament for the treatment of trauma including surgical or accidental trauma, microbial or viral infection, an insect bite, pimples or other skin lesions, a scratch, a stretch mark (striae), a burn, sunburn, a site of body piercing or a melanoma, cancer scar, skin cancer scar, site of biopsy or site of a catheterization event for use in a subject deemed to be at risk of aberrant scar formation following the scar predictability test.

Reference herein to “treatment” and “prophylaxis” is to be considered in its broadest context. The term “treatment” does not necessarily imply that a subject is treated until total recovery. The term “treatment” encompasses the treatment of a healing area before or after a wound. Similarly, “prophylaxis” does not necessarily mean that the subject will not eventually develop some level of fibrosis or some level of inflammation. Accordingly, treatment and prophylaxis include amelioration of the symptoms of a particular fibrotic condition or preventing or otherwise reducing the risk of developing a particular fibrotic or associated inflammatory condition. Such conditions include keloids. The term “prophylaxis” may be considered as reducing the severity or onset of a particular fibrotic condition. “Treatment” may also reduce the severity of an existing fibrotic or associated inflammatory condition.

Topical administration is generally expressed per area of skin or internal epithelial layer. For example, contemplated herein is an amount of follistatin, for example, of from 10 μg to about 100 mg per cm² of skin or epithelium. Such amounts include 10, 20, 30, 40, 50, 60, 70, 80, 90 and 100 μg per cm² of skin or epithelium as well as 100, 200, 300, 400, 500, 600, 700, 800, 900 and 1000 μg per cm² of skin as well as 1, 20, 30, 30, 40, 50, 60, 70, 80, 90 and 100 μg per cm² of skin or epithelium. In an embodiment, the amount of follistatin is expressed in alternative units. Hence, 0.1 nM to 100 nM of follistatin per cm² skin or epithelium may be administered which includes 0.1, 0.2, 0.3, 0.4, 0.5, 0.6, 0.7, 0.8, 0, 9, 1 nM, or 10, 20, 30, 40, 50, 60, 70, 80, 90, 100 nM follistatin per cm² may be employed. Treatment may be daily or weekly or monthly or for as much time as is required to affect a successful clinical outcome, whether this be total prevention of the fibrotic condition such as a keloid or its mitigation to a level which is clinically manageable.

The present disclosure further enables a diagnostic kit comprising agents which detect activin levels in in vitro dermal fibrosis. This may enable slow release or sustained release of the follistatin over time (e.g. for hours to days to weeks to months).

Further enabled herein is a therapeutic kit comprising an activin inhibitor in a medium which permits slow or sustained release of the follistatin over time in or on a subject in need of treatment. This also applies to PB-01 or an inhibitor of CREB protein or a PGE2 inhibitor. In an embodiment, taught herein is a topical or injectable composition comprising an activin inhibitor or a functional variant or isoform thereof in a medium which permits slow or sustained release of the activin inhibitor over time at or near the site of a wound in or near a subject. Hence, the present invention has utility in a range of conditions or therapeutic protocols such as in plastic surgery, cancer surgery, general surgery, catheterization, biopsies, burn management, infection management, immunotherapy and chemotherapy and radiotherapy.

Reference herein to a “gene, miRNA and/or protein expression profile” does not preclude the measurement of other indicators of activin-mediated signaling. Such indicators show the level of sensitivity of a biopsy of dermal fibroblasts to different concentrations of activin over time. Other indicators include mRNA and RNA fragments.

### EXAMPLES

Aspects disclosed herein are further described by the following non-limiting Examples.

### Example 1

### Activin Sensitivity Testing for Predicting Scar Formation

A protocol is developed to assist clinicians in identifying patients at risk of developing hypertrophic or keloid scars and providing treatment regimens to improve wound repair and reduce scar formation.

It is proposed, based on the subsequent examples, that activin stimulation will trigger autoregulation of activins in dermal fibroblasts of patients likely to develop hypertrophic and keloid scarring after surgery.

The protocol comprises pre-surgical in vitro assessment of the activin sensitivity of a sample of dermal fibroblasts from a skin biopsy using the following steps:
1) Collection by the surgeon of a 2 mm skin biopsy.
2) Transfer of the biopsy to the laboratory for assessment.
3) Tissue from the biopsy will be cultured in vitro and treated with various concentrations of activin and tested for a time-related clearance of activin using measures of gene, miRNA and/or protein expression.
4) Based on the responses of the tissue to this scar predictability test, the likelihood of significant scarring post-surgery is determined and this information used to decide on the appropriate method of treatment.
5) If this test shows that activin sensitivity of the tissue is high, the following recommended treatments: a TGF-β antagonist (including a TGF-β1, 2 or 3 antagonist) or activin inhibitor such as AP-1, follistatin, PB-01, an inhibitor of CREB protein or a PGE2 inhibitor.

It is expected that the scar predictability test will clearly indicate patients at risk of hypertrophic and keloid scarring and that the recommended treatments will either prevent or reduce the likelihood of significant scar formation. This technique also allows clinicians to assess the response to treatment in cases where scar formation is normal such as burns, acne, psoriasis, keloids, etc.

### Example 2

### Activin Signaling Pathway in Keloid Pathogenesis

Keloids are known as benign tumors caused by fibrosis during and after wound healing resulting in a symptomatic disfiguring scar. Wound healing is a complex process that involves the actions of various cytokines including activins. Activins are members of the transforming growth factor-β superfamily, and are significantly upregulated during wound healing. Over-expression of activins accelerates wound healing but increases fibrosis at the wound site. Although many studies have investigated the etiology and clinical characteristics of keloid disease, molecular mechanisms of keloid progression remain poorly understood.

Normal and keloid tissue samples were collected from 11 patients and were used to establish primary fibroblast cultures. Using these fibroblasts, qRT-PCR for relative gene expressions and enzyme-linked immunosorbent assay (ELISA) for relative protein expressions were performed. Fibroblasts were also treated with 200 pM activin for 24 hours to examine the effects on fibrosis-related genes.

Results show that activin A gene expression was significantly upregulated in keloid fibroblasts compared to normal fibroblasts. Consistent with the upregulation of gene expression, activin A protein levels in keloid fibroblasts were significantly higher than in normal controls and high levels of activin A protein were also detected in culture medium in vitro. Connective tissue growth factor (CTGF), a gene associated with fibrosis was significantly upregulated in keloid fibroblasts compared to normal fibroblasts. After activin A treatment for 24 hours, activin A and CTGF gene expressions were significantly upregulated in both normal and keloid fibroblasts.

The data show that local production of activins from keloid-derived fibroblasts has a causative association with keloid disease progression.

### Example 3

### Activins as a Causative Factor in Keloid Pathogenesis

Normal and keloid tissue samples were collected from 11 patients and used to establish primary fibroblast cell cultures as described in Example 1. Relative expressions of fibrosis-related genes were examined using qRT-PCR. Protein levels of activin and follistatin, an activin-antagonist, were also measured by ELISA and radioimmunoassay in lysates of dermal fibroblasts. Furthermore, keloid fibroblasts were treated with 1 nM follistatin for 24 hours to examine the effects on fibrosis-related genes.

Results show that activin gene expression was significantly upregulated in keloid fibroblasts compared to normal fibroblasts. Consistent with gene expressions, activin A protein levels in keloid fibroblasts were significantly higher than in normal controls and high production of activin A protein was also detected in medium in vitro. CTGF was significantly upregulated in keloid fibroblasts compared to normal fibroblasts. After follistatin treatment, CTGF gene expression was significantly decreased.

Local production of activin from keloid-derived fibroblasts has a causative association with keloid disease progression. The action of follistatin in suppressing activin and CTGF gene expression includes a role for this protein in treating keloid and other fibrotic diseases.

### Example 4

### Follistatin as a Novel Treatment of Keloid Pathogenesis

Normal and keloid fibroblasts were isolated and cultured from a patient. Relative expressions of fibrosis-related genes were examined using qRT-PCR. Protein levels of activin and follistatin were also measured by ELISA and radioimmunoassay in dermal fibroblasts as described in Example 2. Furthermore, keloid fibroblasts were daily treated with 1 nM follistatin for 1, 3 and 5 days to examine the effects on fibrosis-related genes.

Keloid fibroblasts highly produced activin A gene expression through activin autocrine pathway. These activin effects were gradually stimulated during in vitro cell culture. After 1 nM follistatin treatment for 24 hours, activin A gene expression was significantly decreased compared to normal fibroblasts. During five days of follistatin treatment, activin A and its downstream target, CTGF, gene expression were significantly decreased. At day 5 of follistatin treatment, CTGF gene expression in keloid fibroblasts was similar to normal fibroblasts.

Keloid disease is correlated with local production of activin A (Example 1). The action of follistatin in suppressing activin A and CTGF gene expression indicates a role for this protein in treating keloid and other fibrotic diseases.

### Example 5

### Cutaneous Delivery of Follistatin to Treat Fibrosis

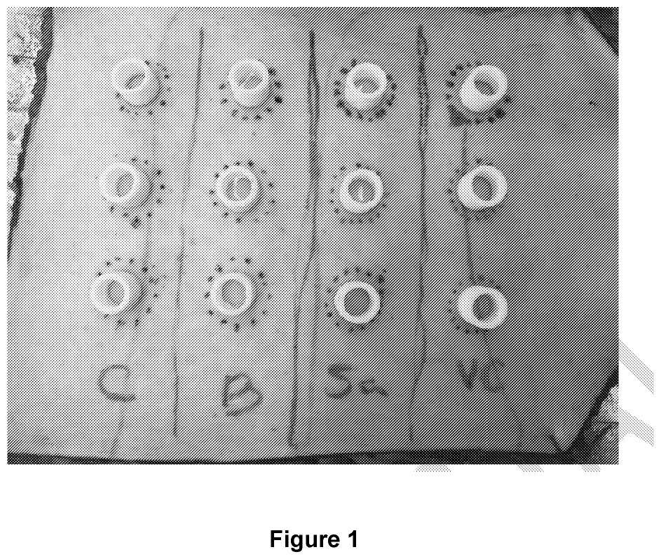

Primary fibroblast cell cultures were established from normal and various fibrotic disease human tissues (such as scars, burn scars, keloids, Dupuytren's disease, scleroderma, eczema, psoriasis) obtained at surgery. These were used to examine the effectiveness of human follistatin isoform 288 (FST288) [PB-01] in identifying, reducing and controlling the expression of inflammatory and fibrotic genes and proteins. Studies were particularly focused on two common human fibrotic diseases—keloid and Dupuytren's contracture. Examples 1 to 3 showed a significant upregulation of the activin A gene and its protein, both inside the cells and secreted in culture medium, compared with control fibroblasts and a significant downregulation of activin A expression of the gene and its protein after treatment with follistatin. Activin A is a well characterized protein that is involved in the inflammatory response and fibrosis. The effectiveness of follistatin in controlling activin A gene expression and secretion in normal and keloid dermal fibroblasts from a patient with keloid fibrotic disease is shown in FIG. 1.

This study has also allowed the examination of the expression patterns of a broad spectrum of inflammatory and fibrosis genes in normal and disease-related fibroblasts in vitro and measure their respective protein levels, for example, follistatin, IL-6, and activins A and B, both inside the cell and in cell culture medium

In this Example, follistatin is formulated in a liquid and gel nano-sized topical delivery medium designed to enable passage of molecules through the skin (Lyotropic Delivery systems, Jerusalem, Israel). The “LDS nano-channel system” provides an effective loading, storage and transcutaneous delivery method for the transport of follistatin into human skin. It is referred to herein as the “nano-channel system”.

The process initially required optimization of the nano-channel system for follistatin isoform 288 (FST288). This involved trial loading nano-channel system with GMP quality FST288 (Paranta Biosciences, Melbourne Australia) and successful demonstration of the formulation stability under different storage conditions, FST288 solubility, and successful release of the follistatin without changing its biological function.

Following the successful optimization of the formulations, known doses of FST288 were loaded into the nano-channels which were then sent as three liquid formulations and one gel for application to live human skin freshly harvested at surgery under human research ethics approval.

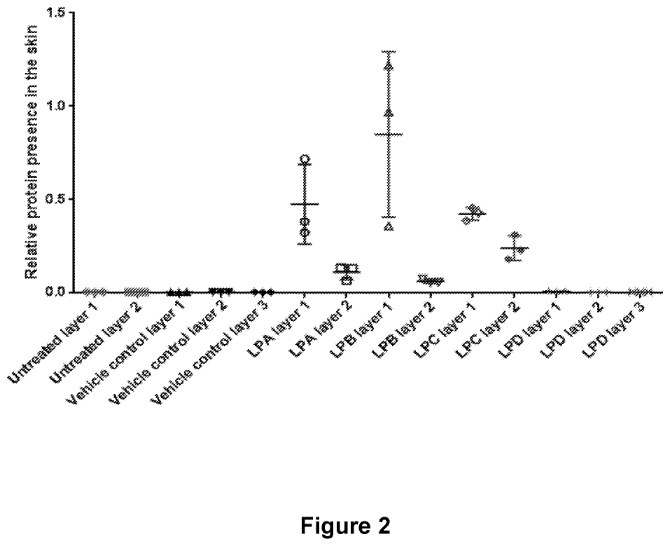

In this trial, fresh live human skin was obtained from three patients during surgery. Immediately following excision from the patient, while maintaining normal skin tension, a series of Teflon rings (10 mm diameter) were glued equidistant from each other to the skin surface (FIG. 2) and the formulations were loaded by micropipette (200 uL) into each Teflon ring. To prevent evaporation, the Teflon rings were covered with a paraffin film. The skin was kept at room temperature and the cutaneous surface of the skin was exposed to the follistatin-loaded liquid or gel nano-channels for up to 48 hours.

The formulation (liquid or gel) in each Teflon ring was replaced with 200 uL of fresh product at 24 hrs. Saline and unloaded nano-channels were used as controls to estimate the natural content of FST288 in the various sections of the skin. All processes were performed in a clean environment. After 24 or 48 hrs exposure, the Teflon rings were emptied by micropipette and any residual fluid was removed by gently wiping the skin surface with sterile gauze. The skin surface was then rinsed carefully with saline to remove any surface contamination of FST288 from the nano-channels. Initial attempts to harvest skin layers from the first experiment using an electric dermatome were only able to produce two layers of about 300 micron each; more consistent thin layers of the experimental skin were then obtained using a hand held dermatome. The second and third skin specimen were separated with a hand held Humby dermatome which allowed a more precise splitting of skin into up to four consistent sequential layers from superficial to deep: superficial epidermis, deep epidermis, superficial (papillary) dermis and deep (reticular) dermis.

This splitting technique provided consistent separate layers of skin which were used to determine the depth of penetration of FST288 into the skin from the nano-channel formulation. A piece of full thickness skin was removed at the end of each experiment from each skin sample and fibroblasts were cultured to confirm that the skin was still viable. A standard protein extraction method was used to extract FST288 from each skin layer and the level of FST288 in each layer from the controls and treated skin was measured using a radioimmunoassay. The amount of FST288 was normalized against the total protein in the extract. Protein extracts from control controls and vehicle controls skin samples were used to measure and account for endogenous levels of FST288 in the skin layers.

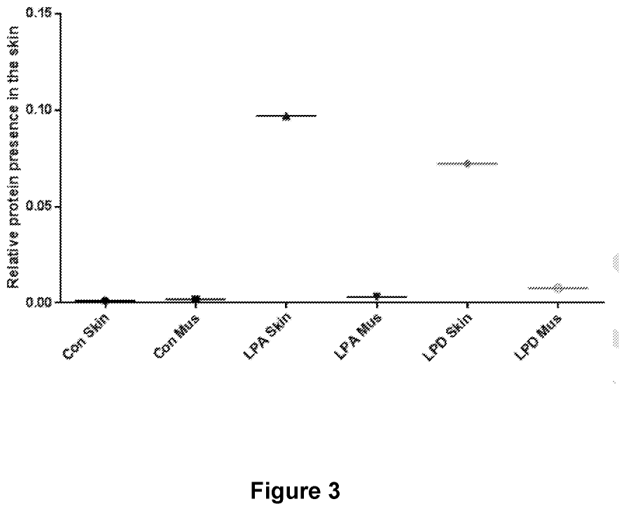

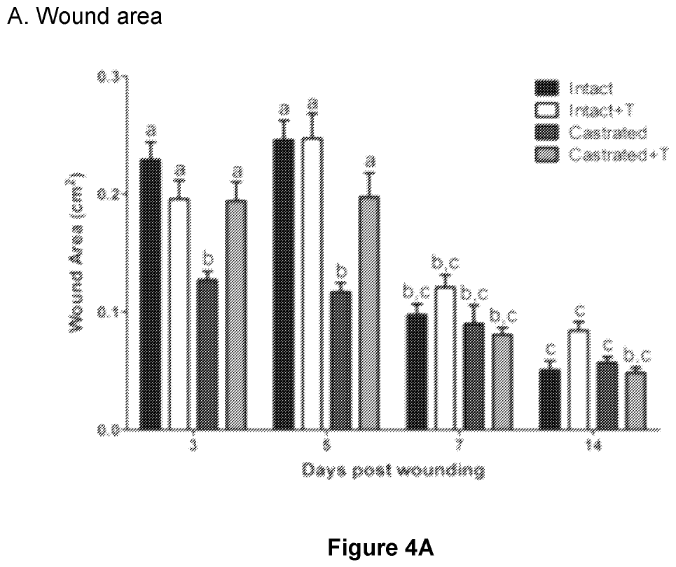

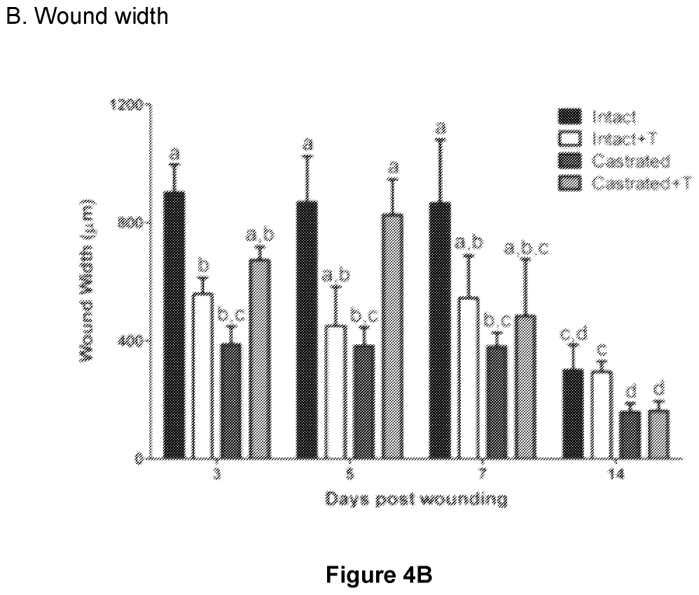

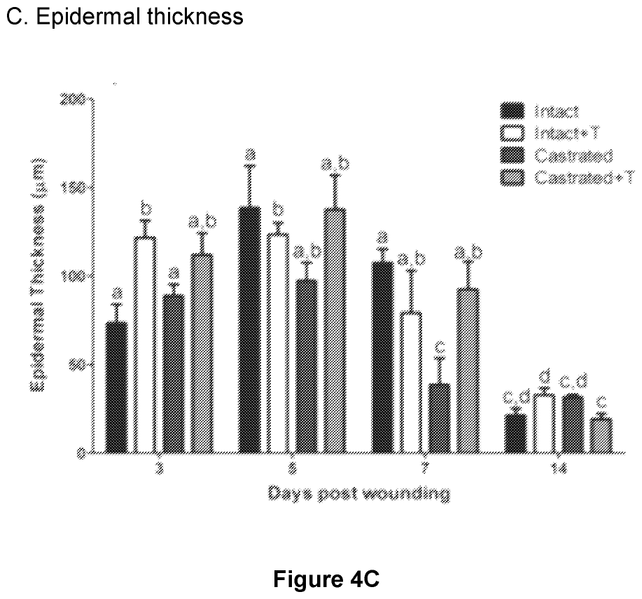

Data obtained from this study using three liquid nano-channel formulations (LPA, LPB and LPC) and one nano-channel gel (LPD) on three different patient skin samples confirmed the hypothesis by showing that both the liquid and gel nano-channels successfully stored and then transported FST288 into the epidermis and dermal layers of human skin taken from the abdomen during abdominoplasty and eyelid during blepharoplasty (FIGS. 3 and 4).

LPB was the most successful liquid formulation for transcutaneous movement of FST288 through the skin layers but in all preparations the relative amounts of FST288 that entered the dermal layers, especially the deep dermal layer was less than the levels in the epidermis. Successful culture of fibroblasts from skin samples after experimentation showed that human skin harvested and used over periods up to 48 hours for these experiments remained viable. The current measure of FST288 that entered the various layers of skin during the experiments is the amount of FST288 relative to the total protein extracted from each sample using a normalization protocol.

This Example establishes a technique to apply a volume of nano-channels containing a known content of FST288 on to live human skin, and to measure the cutaneous penetration of FST288 into the skin layers and this is compared with saline (control controls) and unloaded nano-channels (vehicle controls) applied in a similar manner to the skin. The saline and unloaded nano-channel are designed to control for the presence of endogenous FST288 in the skin. A suitable technique is established to harvest thin layers of treated skin from superficial epidermis to deep (reticular) dermis using a hand held dermatome under strict surgical conditions.

This study has allowed the measurement of FST288 in the different skin layers and to demonstrate the movement of FST288 from the nano-channels formulations into the skin layers. Results confirm that the nano-channels formulation effectively transport FST288 through human skin.

### Example 6

### Human Keloid Fibroblasts Produce High Levels of Activin B

In this Example, a high level of activin B gene and protein expression is found in dermal fibroblasts from a patient with keloid disease.

A 21 year-old Caucasian woman with an unremarkable medical history was selected who presented with a benign tumor on the right ear lobe and to a lesser extent on the left ear lobe. Six years earlier she had undergone uneventful bilateral earlobe piercing and 12 months prior to presentation she noted the onset of an itchy, tender and sometimes painful nodule growing in both ear lobes, which was worse on the right side. A diagnosis of ear lobe keloid was made and surgical excision was undertaken removing 13×7×3 mm and 15×4×9 mm, anterior and posterior of the right earlobe respectively. No recurrence of the earlobe keloid has been reported from the patient since surgery. Other relevant history included the presence of a normal back scar following the surgical removal of a benign naevus, other helical rim and umbilical piercings without keloid formation and the presence of tattoos which had healed normally.

Keloid tissues were fixed in 10% v/v formalin, embedded in paraffin wax, sectioned at a thickness of 5 μm and stained with either haematoxylin and eosin (H&E) or Masson's trichrome stain. Histological examination revealed a thickened, flattened epidermis with a very thick papillary dermis compared to normal and no evidence of malignancy. In these sections, an increased cell number was observed in the papillary dermis which correlated with the presence of high levels of collagen in keloid tissue as shown by extensive Masson trichrome staining in tissue sections.

Gene expression studies of in vitro cultured keloid fibroblasts from the patient showed high levels of both activin A (INHBA) and B (INHBB) genes compared to fibroblasts from normal skin samples (n=4). High INHBA and INHBB were directly related to a high content of activin A and B proteins in cell lysate. Moreover, high activin A expression in these keloid fibroblasts was correlated with high levels of activin A in serum-free medium. Fibroblasts from the keloid patient also secreted large amounts of activin B into serum-free medium but activin B was not detectable in medium from cultured normal fibroblasts.

Hence, the 21 year-old Caucasian girl with ear lobe keloids whose fibroblasts produced an expected high expression of activin A but also especially and unusually high amounts of activin B. Activins have many functions in wound healing and fibrosis and activin A has been shown to stimulate cell proliferation and differentiation during wound repair. Keloid fibroblasts have been reported to produce 29 times higher activin A levels than normal fibroblasts (Mukhopadhyay et al. (2007) Am J Physiol Cell Physiol 292:C1131-1338). In mice, activin A and B mRNA were significantly upregulated within seven days of wound healing (Hubner et al. (1996) Dev Biol 173:490-498) and transgenic mice, which have overexpression of activin A, showed increased wound healing (Mung et al. (1999) EMBOJ 18:5205-5215). This high level activin A gene expression was also found in keloid fibroblasts from the patient and the high INHBA gene expression was positively correlated with high levels of activin A protein in the lysate of fibroblast and in activin A protein secreted in vitro in cultured fibroblast medium. Importantly, activin A secreted by fibroblasts may also affect other cells located in human skin. These observations show a clear and important link between the fibrotic and wound repair activity of keloid fibroblasts and activin A.

Although activin B has similar functions to activin A few studies have focused on the relationship of activin B to fibrotic diseases (Hedger et al. (2011) Vitam Horm 85:255-297). This may be due to the very low levels of activin B protein that have been measured previously in normal and keloid dermal fibroblasts. This study confirmed extremely low basal levels of activin B in cell lysate of normal fibroblasts from the keloid patient but showed unusually high and significantly elevated levels of activin B in cell lysate from her keloid fibroblasts which were positively correlated with significantly upregulated expression of the activin B gene (INHBB).

Keloid is a difficult clinical entity to control because at present there is no effective cure for this symptomatic disfiguring tumor. Injections of steroid, or similar agents, radiotherapy, pressure therapy and repeated surgical excision have all been advocated with variable long term results, and often long lasting psychosocial impacts on patients. In this Example, a patient is described with keloid developing several years after injury who has not only extremely high levels of activin B but also activin A gene expression and protein secretion from dermal keloid fibroblasts in vitro. This unusually high level of both activin B gene and protein expression in the keloid fibroblasts indicates the possibility that an extrinsic factor is involved in the development of the keloid in this patient. These observations add support to the likely causes of keloid being multifactorial and suggest that treatment of this and other keloids by a powerful inhibitor of activins may provide an effective pharmaceutical approach to control and shrink keloids. Hence, enabled herein is a method for the treatment of a wound or skin condition in or on a subject, the method comprising topically applying to the wound or site of skin condition and/or its surrounding region, follistatin or a functional variant or isoform thereof for a time and under conditions sufficient to reduce the effects of fibrosis or an inflammatory condition associated with fibrosis on the wound or skin condition. In an embodiment, the wound is selected from the group consisting of injury or surgical trauma, site of a Dupuytren's disease, site of a microbial or viral infection, an insect bite, pimples or other skin lesions, area of psoriasis or scleroderma, eczema, a scratch mark, stretch mark (striae), acne, a burn, sunburn, a site of body piercing as well as melanomas and cancer scars such as skin cancer scars as well as hypertrophic scars. In an embodiment, the wound is exacerbated by a condition selected from the group consisting of type 1 or 2 diabetes, skin ulceration, obesity, aging, coronary heart disease, peripheral vascular disease, wound infection, cancer, immunosuppression and the effects of radiation or chemotherapy as well as the site of catheterization or the site of a biopsy.

### Example 7

### Activin a, Follistatin and Inflammation Responses to Dermal Wounding are Reduced and Wound Repair is Accelerated in the Absence of Testosterone

In this Example, the hypothesis that testosterone and its withdrawal affects wound healing by modulating cutaneous levels of activin A and follistatin is investigated. The aims of the study were: (i) to compare circulating and cutaneous levels of activin A and follistatin during cutaneous wound repair in adult male mice in the presence (intact) and absence (castrated) of androgens; (ii) to examine the effects of exogenous administration of testosterone on the cutaneous concentrations of activin A and follistatin in skin during wound repair in intact and castrated adult male mice (iii) to determine if the modulation of activin A and follistatin by exogenous testosterone affects the levels of the pro-inflammatory markers IL-6 and TNF-α and the number of infiltrating leukocytes during wound repair.

### Animals

Six to nine week-old male Balb/cJASMU mice obtained from the Monash University Animal Services, Monash University, Clayton, Victoria, Australia, were housed prior to and during experiments under the following conditions: temperature range 21° C. to 24° C.; light cycle 12 hours light: 12 hours dark. All mice had access to food and water ad libitum.

Animals were anesthetized by intra-peritoneal injection of ketamine/xylazine (Ketamine: 90 mg/kg body weight, Parnell Australia Pty Ltd, NSW, Australia; Xylazine: 10 mg/kg body weight, Troy Laboratories Pty Ltd, NSW, Australia). Anesthesia was used for all surgical procedures (gonadectomy, placement of silastic implants, wounding). Levels of anesthesia were checked by tail-pinch and pedal reflex. A long-acting systemic analgesic (Carprofen 5 mg/kg, Lyppard Australia, Melbourne, Australia) was administered to provide post-surgery pain relief.

Six week-old male mice were castrated under anesthesia three weeks prior to the studies on wound healing. The testes were gently pushed through the inguinal canal into the abdominal cavity and exposed through a small (0.5 cm) ventral midline abdominal incision. Each testis was gently dissected from its epididymis and removed after ligation of the testicular vasculature. All incisions were closed using two interrupted 5/0 silk sutures (Johnson & Johnson Medical, NSW, Australia).

### Silastic Implants

Implants were prepared by cutting medical grade Silastic (polydimethylsiloxane) tubing (1.5 mm inner diameter, 2.3 mm outer diameter, Aunet Pty Ltd, WA, Australia) to the desired length (1 cm long) and sealing one end with Multi-Purpose Sealant (Dow Corning RTV Sealant). After 24 hours each tube was packed with either crystalline testosterone (Sigma #T-1500) or left empty (vehicle implant). The open end was then sealed and implants were allowed to dry for at least 24 hours prior to surgical implantation. The implant size was considered to be the length of tubing containing testosterone. Prior to subcutaneous insertion, implants were sterilized in absolute ethanol for 10 minutes which also removed any androgen adhering to its external surface. Each testosterone or vehicle implant was inserted subcutaneously via a 5-mm nape incision in intact and castrated male mice three weeks before the wound healing experiments and the incision site was closed with 5/0 sutures.

### Wounding Experiments

Under anesthesia, the dorsal flanks of each 9 weeks old mouse (n=6/group) were carefully shaved and cleansed with 70% v/v ethanol. To create wounds, full-thickness, parasagittal linear incisions (1 cm long) were made through the skin and underlying the panniculus carnosus muscle on each flank and each wound was closed using two 5/0 silk sutures placed 3 mm from each end of the wound. Post operatively, all animals received analgesia to minimize pain, were allowed to recover on a heated pad and then housed in groups of four animals per cage. Incisions were examined and assessed on days 3, 5, 7 and post-wounding.

### Serum Collection

Blood samples were collected between 10:00 and 10:30 am by cardiac puncture at different time points (0d, 3d, 5d, 7d or 14d) according to the experimental protocol. The samples were allowed to coagulate for 30 minutes then centrifuged at 10,000 g for 10 minutes to collect serum which was stored at −20° C. until assayed.

### Tissue Collection and Processing

Following exsanguination, anesthetized animals were killed by cervical dislocation. Full-thickness specimens of the skin wound, consisting of the entire wound scar surrounded by a border of about 3-mm, were excized and bisected. Half of each sample was frozen at −80° C. and the other half was fixed in 4% v/v paraformaldehyde (Sigma-Aldrich, NSW, Australia) and processed for histology. Unwounded skin was obtained from the dorsum of the trunk near the tail of each animal to determine protein levels.

The testes, epididymides, and seminal vesicles from animals with and without testosterone were dissected, cleaned of associated fat and connective tissue, and weighed. Weights were then used to compare the effectiveness of testosterone replacement on reproductive organs of the castrated males, and also to examine any differences in organ weights between normal mice with either vehicle or testosterone containing implants. These organ weights are a physiological measure of circulating androgen levels.

### Macroscopic Evaluation

Digital images of each dorsal skin wound were obtained with a standardized focal distance, aperture and exposure time using a Nikon D5000 camera (Nikon, Tokyo, Japan) immediately after the initial incision and at 3, 5, 7 and 14 days post-wounding. Wound areas were measured within the wound margins and the pixel areas were calculated using Adobe Photoshop CS5 (version 12.0, Adobe Systems Inc). The area of erythema was measured in all groups at 3 days and 5 days post-wounding and the area of the wound was measured in all groups at all time-points.

### Histopathological Measurements

Transverse histological sections (5 μm) of skin from the center of each wound were stained with hematoxylin and eosin (H&E) [Harris' Hematoxylin, 1% v/v Eosin, Amber Scientific, Midvale, Wash., Australia] and analyzed by light microscopy. All histological analyses were performed blind without knowledge of the identity of each specimen. Histological sections were scanned for assessment using Aperio ScanScope AT Turbo Scanner (Aperio, CA, USA) and the electronic slides (eSlides) were visualized and analyzed using Aperio ImageScope.

Wound width was calculated by measuring the distance between the unwounded dermis margins at the epidermis-dermis junction (Gilliver et al. (2008) endocrinology 149(11):5747-5757). When the width was <0.2 mm, the wound was considered closed. Reepithelialization was assessed using the following scoring method: 0, absent; 1, present, covering <50% of the wound; 2, present, covering >50% and <100% of the wound; 3, present, covering 100% with irregular thickness; 4, present, covering 100% with regular thickness (Steed et al. (1997) 77(3):575-586). To determine epidermal hyperplasia, the mean distance between the stratum granulosum and the epidermal-dermal junction of each wound site was calculated (10 measurements per section, using a 10× objective). Unwounded skin was used as control.

Sections from day 7 and day 14 wounds were also stained with Masson's trichrome to highlight connective tissue. The area of granulation tissue was measured by defining the area located between the basal surface of the epidermis and the panniculus carnosus below. Collagen orientation was assessed using the following scoring method: 1, basket-weave fibers; 2, basket-weave >parallel fibers; 3, parallel fibers >basket-weave fibers; 4, parallel fibers (Ashcroft and Mills (2002) J Clin Invest 110(5):615-624).

### Immunohistochemical Analysis of Leukocyte Infiltration

CD45, also known as leukocyte common antigen (LCA), was used to detect infiltrating leukocytes as a marker of inflammation (Hermiston et al. (2003) Annul Rev Immunol 21:107-137). Five micron thick sections were cut and placed on Menzel-Glaser Polysine (Registered Trade Mark) slides (Thermo Scientific), air-dried and stored at room temperature. Slides were stained using an automated system (DAKO Autostainer Plus), at room temperature; sections were rehydrated and antigen retrieved with the Dako buffers citrate pH6.1. Slides were peroxidase-blocked in 3% v/v hydrogen peroxide (H₂O₂) in methanol for 10 mins. They were then blocked with CAS-Block (Invitrogen, CA, USA) for 10 minutes, and then incubated with primary antibody (CD45 biotinylated rat anti-mouse antibody, 1:200) for 20 min. Vectastain ABC kit (Vector Labs, Burlingame, Calif.) was subsequently applied for 30 minutes and further colorimetric detection was completed with diaminobenzidine (DAB) for 5 minutes. Sections were counterstained with haematoxylin and slides coverslipped under DePeX.

Leukocyte infiltration was assessed by calculating the average number of CD45+ cells in four random high-power fields (HPF) per tissue section from each group. Data presented reflect the mean total cell count per field from the wound area at days 3 and 5 post-wounding.

### Assays

Serum testosterone levels were measured using a direct radioimmunoassay (RIA) testosterone kit (IM1119—Immunotech, Marseilles, France) according to the manufacturer's instructions, using I¹²⁵-labeled testosterone as a radioactive tracer. The antibody used in the immunoassay is highly specific for testosterone with extremely low cross-reactivity (<0.75%) for related molecules such as 5a-dihydrotestosterone or 44-androstenedione. The assay sensitivity for serum testosterone was 15.63 pg/ml and the intra-assay variation was 7.9%. All samples from one experiment were measured in the same assay.

Frozen samples of wounded and unwounded skin were homogenized (Janke & Kunkel Ultraturrax T25 homogenizer, IKA Labortechnik, Staufen, Germany) in 1% v/v protease inhibitor (Calbiochem, San Diego, Calif.) and the homogenates were centrifuged at 4° C. to remove debris prior to assay.

Serum and skin follistatin levels were measured by RIA using human recombinant follistatin 288 as standard and as tracer following labeling with I¹²⁵ (O'Conner et al. (1999) Hum Reprod 14(3):827-832). The assay sensitivity for serum follistatin was 1.52 ng/ml, and the intra-assay variation was 5.4-5.7%.

Activin A levels were measured in serum and skin using a specific ELISA and human recombinant activin A as a standard, according to manufacturer's instructions (Oxford Bioinnovations, Cherwell, Oxfordshire, UK) [Knight et al. (1996) J Endocrinol 148(2):267-279]. The assay sensitivity for serum activin A was 11 pg/ml, with an intra-assay variation of 4.6-7.5% and an inter-assay variation of 10.6%.

Cutaneous levels of IL-6 and TNF-α were measured in skin samples using ELISAs according to the manufacturer's instructions (R&D Systems Inc, MN, USA). The sensitivity of the IL-6 ELISA was 5.97 pg/ml; the intra-assay variation was of 3.4% and the inter-assay variation was 4.1%. Mouse recombinant was used as a standard for the TNF-α assay; the sensitivity of the ELISA was 9.04 pg/ml; the intra-assay variation was of 12.8% and the inter-assay variation was 12.7%. The concentrations are expressed per mg of tissue (wet weight).

### Statistical Analysis

Data are expressed as mean±SEM. Statistical analysis was performed using SPSS version 15 (IBM, Armonk, N.Y., USA). Data were analyzed using two-way ANOVA. Basal (day 0) comparisons between intact and castrated groups were analyzed using Student t-test. Comparisons between groups were analyzed using one-way ANOVA with Tukey's post hoc test. Re-epithelialization and collagen scores were analyzed with Kruskal-Wallis and Mann-Whitney U non-parametric tests. Because of skewed distributions, serum testosterone levels were log-transformed to allow parametric testing. Differences were considered statistically significant at p<0.05.

### Validation of Castration and Testosterone-Replacement Experiments Using Serum Testosterone Levels and Reproductive Organ Weights

Castration of male mice significantly reduced serum testosterone levels (intact males: 14.12±3.2 vs castrated males: 0.04±0.01, p<0.001; Table 2) and the size and weight of the seminal vesicles (0.27±0.01 vs 0.04±0.01, p<0.05) and epididymis (0.05±0.04 vs 0.02±0.02, p<0.05) compared to intact males. Testosterone replacement in castrated males significantly increased serum testosterone levels back to control levels seen in both intact and intact+T males (Table 2) and returned the size and weight of the seminal vesicle (0.42±0.02, p<0.05) and the epididymis (0.05±0.02, p<0.05) to the sizes found in intact males.

Following wounding, reproductive organ weight remained constant in each group until the end of the experimental process.

TABLE 2
Effect of wounding, testosterone replacement and wound repair on serum levels
of testosterone, activin and follistatin between intact and castrated males
at all time-points (Data expressed as mean ± SEM; n = 6 per group)

| | | | | |
|---|---|---|---|---|
| 0 d | 3 d | 5 d | 7 d | 14 d |

Serum Testosterone (ng/ml)

| | | | | | |
|---|---|---|---|---|---|
| Intact | 14.12 ± 3.2 | 8.9 ± 4.7 | 2.4 ± 2.0 | 6.7 ± 2.7 | 9.6 ± 3.7 |
| Intact + T | 5.3 ± 0.4 | 5.9 ± 0.4 | 7.8 ± 0.9 | 6.7 ± 0.4 | 6.3 ± 0.3 |
| Castrated | 0.04 ± 0.01 * | 0.06 ± 0.02 * | 0.1 ± 0.02 * | 0.05 ± 0.01 * | 0.03 ± 0.0 * |
| Castrated + T | 5.7 ± 0.8 * | 5.4 ± 0.5 * | 5.9 ± 0.4 * | 5.2 ± 0.5 * | 4.5 ± 0.1 * |

Serum Activin A (pg/ml)

| | | | | | |
|---|---|---|---|---|---|
| Intact | 96.8 ± 11.7 | 103.8 ± 2.6 | 98 ± 9.1 | 96.1 ± 12.04 | 89.2 ± 2.8 |
| Intact + T | 65.4 ± 6.8 | 85.2 ± 5 | 74.9 ± 6.9 | 92.1 ± 3.8 | 74.6 ± 3.5 |
| Castrated | 71.9 ± 7.5 | 119.7 ± 20.5 | 130.7 ± 21.8 | 78 ± 4.7 | 87 ± 7.8 |
| Castrated+ T | 80.2 ± 7.8 | 65.4 ± 5.8 * | 54.9 ± 7.7 * | 83.8 ± 7.3 | 93 ± 7.7 |

Serum Follistatin (ng/ml)

| | | | | | |
|---|---|---|---|---|---|
| Intact | 3.3 ± 0.2 | 3.1 ± 0.2 | 3.5 ± 0.4 | 3.4 ± 0.2 | 3.2 ± 0.1 |
| Intact + T | 3.6 ± 0.03 | 3.8 ± 0.3 | 3.8 ± 0.1 | 3.8 ± 0.4 | 3.6 ± 0.2 |
| Castrated | 3.6 ± 0.2 | 3.4 ± 0.1 | 4.8 ± 0.9 | 3.8 ± 0.2 | 4.1 ± 0.3 * |
| Castrated + T | 4.3 ± 0.5 | 4.1 ± 0.3 | 3.9 ± 0.2 | 4.5 ± 0.3 | 4.5 ± 0.5 |

* p < 0.5 vs corresponding intact males; +p < p. 50 vs corresponding castrated males.
Values with no superscript are not signifacantly different p < 0.5.
Intact: intact males treated with vehicle; Intact + T: intact males treated with vehicle; Castrated: castrated males treated with vehicle; Castrated + T: castrated males treated with vehicle.

Erythema was observed around the wound site in all animals at day 3 post-wounding. In the vehicle-treated groups, intact males (intact) had an increased area of erythema compared to the castrated males (castrated) at day 3 post-wounding (0.51±0.05 vs 0.16±0.02 cm2, p<0.001). When testosterone was replaced in castrated males (castrated+T), the area of erythema was significantly increased compared to the castrated group (0.16±0.02 vs 0.42±0.03 cm2, p<0.001). No significant difference was observed between the intact+T and castrated+T or between the intact and intact+T males.

Scabs were present along the external surface of wounds at days 3 and 5 post-wounding in the intact group, however, scabs had been lost in the castrated group by day 3 leaving only a fine scar. When testosterone was replaced in castrated males, scabs were observed at both days 3 and 5 similar to those in intact males, and scars were thicker and more marked than those of the castrated group.

The wound area in intact males treated with vehicle was similar at days 3 and 5 post-wounding, with a significant decrease at day 7 (p<0.05). In the castrated group, the wound area was similar between days 3, 5 and 7 post-wounding with a further decrease at day 14 (p<0.05). Between groups, wound area was greater at days 3 and 5 post-wounding in the intact group compared to the castrated group. Castrated animals treated with testosterone had an increased wound area at days 3 and 5 post-wounding compared to those vehicle treated, with an area similar to the intact males (FIG. 4A).

Histological evaluation of the wounds at day 3 post-wounding showed the presence of a layer of debris on the wound surface in all groups. Although re-epithelialization had begun in the wounds of intact and castrated+T males by day 3, with keratinocytes migrating underneath the scab, this process was not yet complete in either group. In contrast, wound re-epithelialization was completed by day 3 in the castrated group. By day 5 post-wounding, re-epithelialization was complete and granulation tissue had started to form in the wounds of the intact and castrated+T groups.

Wound width showed no change in the intact group until day 14, when it decreased significantly compared to days 3, 5 and 7. In the castrated group, there was also no change in the wound width at days 3, 5 and 7 post-wounding, but there was a significant decrease at day 14. However at each time point wound width was significantly less in the castrated groups than in the intact groups (FIG. 4B).

Testosterone replacement in the castrated males resulted in significantly increased wound widths at days 3 and 5 post-wounding compared with the castrated group, with a similar pattern of wound repair to that observed in intact males. Testosterone treatment of intact males decreased wound width at day 3 post-wounding compared to the intact group (FIG. 4B).

The thickness of the epidermis was significantly increased at day 3, 5 and 7 post-wounding in the intact group but returned to normal thickness by day 14. The castrated group also showed an increased epidermal thickness at days 3 and 5 post-wounding, but this returned to pre-wounding thickness by days 7. Comparison of epidermal thickness in both vehicle-treated groups showed a significantly increased epidermal thickness at day 7 post-wounding in intact males. The castrated+T group showed an increased thickness of the epidermis at days 3, 5 and 7 post-wounding and a return to pre-wounding thickness by day 14; a pattern similar to that observed in intact males (FIG. 4C).

In summary, testosterone delayed wound closure in male mice, increasing the area of erythema post-injury and delaying the recovery of the epidermal architecture.

### Effects on Serum Levels of Activin A and Follistatin

Castration of male mice significantly decreased serum testosterone levels with no significant effect on serum activin or follistatin levels. Following wounding of the skin, there was no significant differences in serum activin between intact and castrated males treated with vehicle. However, serum follistatin had significantly increased by day 14 post-wounding in castrated males compared to intact males (p<0.05). Testosterone replacement in castrated males decreased serum activin levels at both days 3 and 5 post wounding compared to the castrated+Vh group (p<0.05, Table 2).

Testosterone Increased Cutaneous Levels of Activin A after Wounding of the Skin

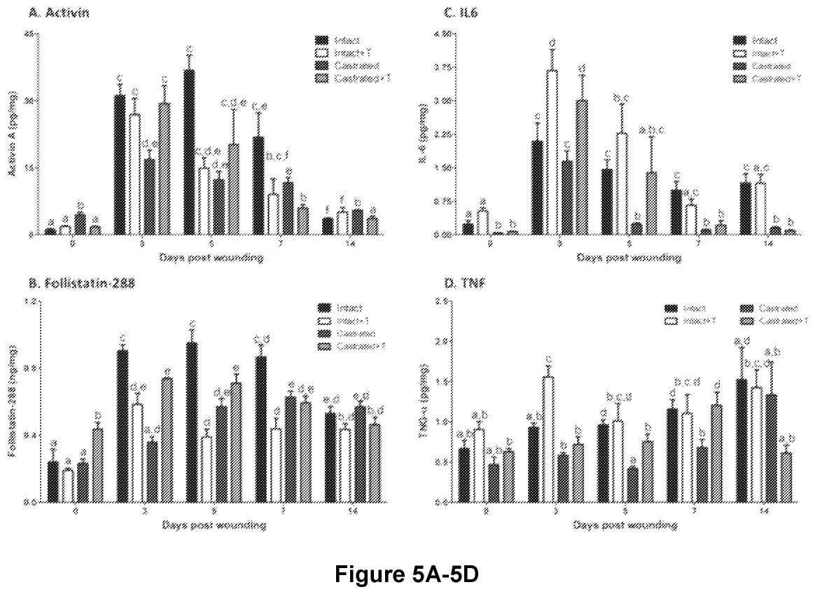

After wounding of intact male mice, there was a 29-fold increase in activin A skin levels by day 3, which remained elevated to day 7; a significant decrease in activin A had occurred by day 14 but concentrations remained above basal levels. In the castrated group, cutaneous activin A increased 4-fold by day 3 post-wounding and remained elevated up to day 7 but returned to base levels by day 14. In both vehicle groups, basal levels of activin A in unwounded skin taken at sites distant to the wound area were significantly decreased in the intact males compared to the castrated group (1.05±0.32 vs 4.41±0.61 pg/mg; p<0.05). However, after wounding, activin A skin levels in intact males were twice those of castrated males at day 3 and 3-times higher at day 5 (FIG. 5A).

Testosterone replacement in castrated males stimulated a 17-fold increase in cutaneous levels of activin A at day 3 post-wounding, which were twice those of the castrated males. However, by day 7 and 14 post-wounding, activin levels were higher in the castrated group. No significant differences were observed between the castrated+T males and the intact males, nor between the intact and the intact+T group (FIG. 5A).

Following wounding in intact male mice, follistatin increased 4-fold by day 3 post-wounding then declined by day 7 but remained above basal levels at day 14. In the castrated males, follistatin did not increase until day 5 post-wounding, but then remained elevated until day 14 without returning to baseline. Comparing the two vehicle control groups, follistatin levels were significantly higher at days 3 and 5 in intact males compared to the castrated males (p<0.05, FIG. 4B).

Although testosterone replacement in castrated males increased cutaneous levels of follistatin as early as day 3 post-wounding, these levels had returned to baseline by day 7. In the two castrated groups, follistatin levels in skin were significantly higher at day 3 post-wounding in the testosterone replacement group compared to the vehicle group (p<0.001). When testosterone was administered to intact males, there was an increase in follistatin at day 3 post-wounding with no further decrease through time. However, these levels were still significantly lower than in intact males that received vehicle (p<0.05; FIG. 4B).

Effects of Testosterone on Inflammation after Wounding of the Skin

At day 3 post-wounding, both intact and castrated males showed increased infiltration of CD45+ inflammatory cells compared to healthy unwounded skin. However, by day 5 post-wounding, the number of cells had significantly decreased in the castrated group whereas there was no change in the number of CD45+ cells in the intact group. When testosterone was replaced in the castrated group, the number of CD45+ cells was significantly increased at both days 3 and 5 post-wounding compared to the castrated group.

### Effects of Wounding and Testosterone on Cutaneous Levels of IL-6 in Intact and Castrated Males

Basal levels of IL-6 in unwounded skin were higher in intact males than in castrates. Following wounding of intact males, there was a significant increase in cutaneous levels of IL-6 at day 3 post wounding (p<0.01) which remained significantly elevated for the duration of the experiment. Castrated males showed a significant increase in IL-6 at day 3 post-wounding (p<0.005). However by day 5 these levels returned to baseline with no further change. These levels were significantly lower in castrated males than in the intact group at days 5, 7 and 14 post-wounding (FIG. 4C).

Testosterone replacement in castrated males stimulated an increase in IL-6 at day 3 post-wounding which was twice that of the castrated group but by day 7, IL-6 levels in castrated+T males had returned to baseline. Although the IL-6 pattern was similar with a significant increase at day 3 post-wounding and returning to baseline by day 7 in castrated+T and intact+T males, the castrated+T males had lower levels of IL-6 in unwounded skin (day 0) and at days 7 and 14 post-wounding. Testosterone treatment of intact males increased basal IL-6 levels, and at day 3 post-wounding IL-6 levels were almost twice the levels of the intact male group. No further differences were observed between these groups (FIG. 4C).

TNF-α levels in skin increased significantly at days 5 and 7 post-wounding in the intact group (p<0.05) but remained constant in the castrated group. Between these two groups, levels were significantly higher at days 3, 5 and 7 post-wounding in the intact males (p<0.05; FIG. 4D).

When testosterone was replaced in the castrated males, TNF-α skin levels significantly increased at day 7 post-wounding (p<0.05). Further, these levels were significantly higher at days 5 and 7 compared to the castrated males. Interestingly, when testosterone was given to intact males, TNF-α skin levels were significantly increased at day 3 post-wounding compared to the intact group (FIG. 4D).

In summary, testosterone caused an increased migration and infiltration of leukocytes to the wound site with increased levels of pro-inflammatory cytokines and an extended the inflammatory phase in male mice.

### Effects of Testosterone on the Wound Dermal Architecture

Collagen fibers in the dermis of intact males treated were disorganized in the early period of wound repair and displayed a predominance of collagen fibers oriented parallel to the epidermis at days 7 and 14 post-wounding. The castrated group presented a predominance of parallel collagen fibers at day 7 post-wounding, similar to the intact group. However, by day 14 there was a predominance of basket weave orientation of collagen fibers, characteristic of a normal dermal structure, in the castrated group, with hair follicles observed at the edges of the wound.

Castrated males treated with testosterone showed immature granulation tissue at the wound site by day 7 post-wounding and collagen fibers had a parallel orientation. The granulation tissue was matured and collagen deposition completed by day 14, but collagen fibers still showed a predominant parallel orientation which differed significantly from the basket weave dermal structure of collagen fibers observed in the castrated group (FIG. 4).

Intact males and intact and castrated males treated with testosterone had a predominance of collagen fibers parallel to the epidermis at days 7 and 14 post-wounding.

This Example demonstrates that testosterone not only regulates the levels of activin A in normal skin, but that it also modifies the activin A response following skin injury, altering the inflammatory process and thereby delaying skin repair. In the absence of testosterone, levels of activin A in the skin increase significantly in response to wounding. However, in the presence of testosterone, a significantly greater increase in activin A levels was observed. Similarly, the levels of pro-inflammatory cytokines such as IL-6 and TNF-α in the skin showed a significantly greater increase in intact male mice than in castrated males indicating an important role for testosterone in establishing the inflammatory response to wounding. Moreover, there was a positive correlation between increases in these pro-inflammatory markers and increased levels of activin A during the inflammatory phase of healing suggesting that testosterone and activin A act together in order to enhance the inflammatory response during wound repair.

In the presence of testosterone, there was an increase in skin levels of both activin A and follistatin, which were significantly higher compared to those of testosterone-deficient male mice. Increased levels of activin A were observed during the inflammatory phase of wound repair, establishing a pro-inflammatory stimulus affecting epidermal structures.

The data show that in day 3 wounds there were increased numbers of CD45+ cells compared to healthy unwounded skin, with elevated levels of IL-6 and activin A in skin, consistent with the inflammatory phase of healing. However, by day 5 post-wounding, the number of CD45+ cells and the levels of IL-6 were decreased only in testosterone-deprived male mice. The present data demonstrate that infiltration of inflammatory cells into the wound area was consistent with an induction of activin A, IL-6 and TNF-α skin levels in all male mice. However, the infiltration of inflammatory cells was significantly reduced in testosterone-deprived males compared to intact male mice with normal levels of testosterone suggesting that testosterone levels establish the required environment for infiltration of CD45+ cells necessary for skin repair and regeneration. This raises again the question of a link between activin A and testosterone to stimulate inflammation at the wound site and delaying wound repair.

This Example shows that testosterone interacts with activin A during the process of wound healing, enhancing inflammation and delaying wound repair. The Example provides further support for the view that activin A is acting as the pro-fibrotic mediator of testosterone by enhancing the inflammatory response and leading to excessive collagen deposition, and the alteration of the dermal structure. Optimal scar development after wounding requires a delicate balance between the influences of androgens, activin A and follistatin. Based on these data, it is suggested that the exogenous administration of follistatin around the wound site in intact male mice will decrease activin A levels and the levels of pro-inflammatory cytokines and result in reduced fibrosis and improved scar formation during wound repair.

### Example 8

### Follistatin, an Antagonist of Activin, as a Novel Treatment in Keloid Disease

Normal and keloid fibroblasts were isolated and cultured in vitro using standard fibroblast cell culture protocols. Relative gene expressions were examined using qRT-PCR. Protein levels of activin and follistatin were also measured in dermal fibroblasts by enzyme-linked immunosorbent assay and radioimmunoassay respectively. Cells were also treated with 100 ng/ml follistatin for 5 days to examine the effects on the expression of fibrosis-related genes.

Keloid fibroblasts displayed elevated levels of activin A gene and protein expression through an activin autocrine pathway. These activin effects were gradually stimulated during in vitro cell culture. After single treatment with follistatin, activin A gene expression in keloid fibroblasts was significantly decreased confirming that the autocrine actions of activins are inhibited by this treatment. Moreover, downstream targets of activins such as connective tissue growth factor (CTGF) declined significantly in keloid fibroblasts compared to controls.

Keloid disease is linked to the local production of activin A. The action of follistatin in suppressing activin A and CTGF gene expression indicates a novel role for this protein in treating keloid and other fibrotic diseases.

### Example 9

### Dupuytren's Disease

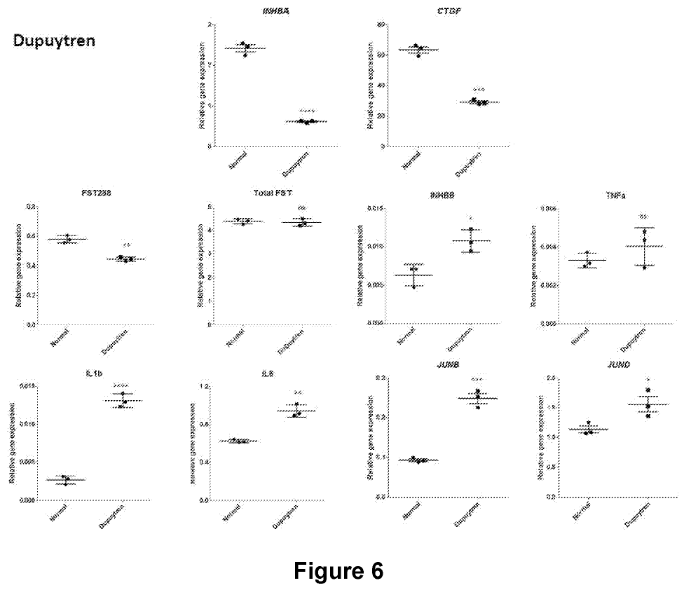

The role of follistatin in the treatment of Dupuytren's disease is shown in FIG. 6.

### Example 10

### Histological Analysis of Keloid Tissue Compared to Normal Control

The histology of keloid tissues was characterized using haematoxylin and eosin (H&E), Masson's trichrome, and Hart's Elastin Stain. H&E showed a thicker epidermis and papillary dermis in keloid tissues compared to normal control skin samples. Keloid tissues had larger numbers of cells present within these tissue layers than in normal tissues. Masson's trichrome stained tissues showed large depositions of collagen in the papillary dermis of keloid tissues compared to those of normal tissues. In contrast, based on Hart's Elastin Stain, normal tissues showed a ubiquitous distribution of elastin fibres whereas relatively few elastin fibres were present in keloid tissues.

### Example 11

### Gene and Protein Expression of Normal and Keloid Fibroblasts

Gene and protein expression was compared in multiple patient samples and also in normal and keloid fibroblasts from a single patient. The single patient comparison between normal and keloid fibroblasts provided a robust complementary intra-patient analysis. Activin A gene (INHBA) expression was significantly upregulated in keloid fibroblasts when compared to normal controls. However, activin B gene (INHBB) expression was not different between normal and keloid fibroblasts and its expression was much lower compared with INHBA. Connective tissue growth factor (CTGF also known as CCN2), a well-known fibrosis related gene, was significantly upregulated in keloid fibroblasts. However, the expression of plasminogen activator inhibitor 1 (PAI1 also known as SERPINE1), which is related to keloid disease, showed wide variation between samples. Consistent with INHBA expression, activin A proteins in cell lysate and culture media were significantly higher in keloid fibroblasts than in normal fibroblasts. Activin B protein was present at very low levels in both normal and keloid fibroblasts. These results were confirmed in normal and keloid fibroblasts from the same site in the single patient. INHBA, CTGF, and PAI1 were significantly upregulated in keloid fibroblasts and INHBB showed significantly lower expression in both normal and keloid fibroblasts. Expression of matrix-related genes, fibronectin (FN1) and tissue inhibitor of metalloproteinases 1 (TIMP1), were also significantly increased in keloid fibroblasts whereas elastin (ELN) was significantly reduced compared to normal controls. To investigate the role of activins in keloid pathogenesis, activin A gene and protein expression was measured for 7 days in keloid and normal fibroblasts. Normal fibroblasts maintained a basal level of activin A gene expression after 3 days onward whereas activin A gene expression continued to increase in keloid fibroblasts for 7 days. Consistent with gene expression, the protein levels of activin A in keloid fibroblasts were significantly increased compared to normal control in both cell lysates and culture media after 7 days. Similarly, CTGF expression was significantly upregulated in keloid fibroblasts after 7 days compared to normal control.

### Example 12

### RNA Sequencing (RNAseq) and Ingenuity Pathway Analysis (IPA)

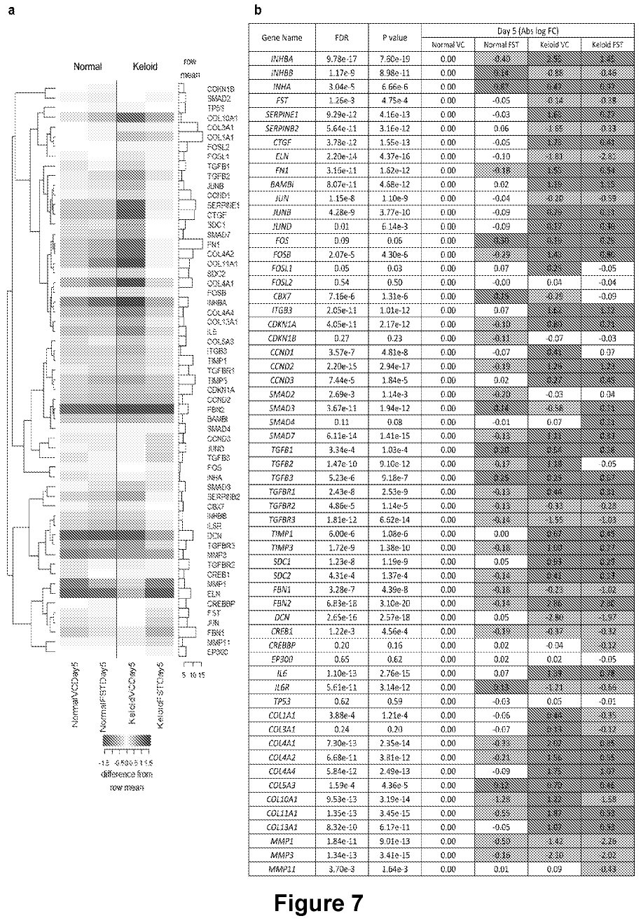

RNAseq was performed with/without a single follistatin treatment on normal and keloid fibroblast cultures on day 1 and day 5 (FIG. 7) to compare the extent of gene expression changes in keloid disease. Consistent with our qPCR and ELISA data, INHBA expression was significantly increased at day 5 compared to day 1 samples (FIG. 7b). These upregulated INHBA expression was diminished by a single follistatin treatment for 5 days (FIG. 7b). Moreover, CTGF and PA1 was also significantly upregulated in keloid fibroblasts on day 5 whereas ELN was downregulated. Some matrix related genes were also significantly upregulated in keloid fibroblasts such as FN1, FBN2, TIMP1, TIMP3, COL1A1, COL3A1, COL4A1, COL4A2, COL4A4, COL5A3, COL10A1, COL11A1, and COL13A1. However, other genes such as DCN, MMP1, MMP3 and MMP11 were significantly downregulated in keloid fibroblasts (FIG. 7b). Notably, after the follistatin treatment, upregulated CTGF and PAI1 were significantly decreased and also other matrix related genes were downregulated (FIG. 7b).

IPA analysis of the transcriptomic data identified the TGFβ pathway as highly enriched in keloid fibroblasts. Fifty-nine genes (FIG. 7b) were identified that showed the most significant upregulated (FIG. 7a) and downregulated expressions (FIG. 7a) in relation to TGFβ signaling in keloid fibroblasts when compared with expression of those genes in normal fibroblasts. Interestingly, Sma- and Mad-related family (SMAD) genes showed similar expression patterns between normal and keloid fibroblasts. Receptor-regulated SMADs (SMAD2 and SMAD3) and the common-mediator SMAD (SMAD4) were not significantly different whereas the antagonistic or inhibitory SMAD (SMAD7) was always significantly upregulated. Expression of negative regulators of TGFβ such as BMP and the activin membrane-bound inhibitor homolog (BAMBI) were, however, significantly upregulated in keloids (FIG. 7b). Expression of the immune-related gene IL6 was also significantly upregulated in keloid fibroblasts whereas the tumor suppression gene TP53 was not altered in normal and control fibroblasts. Furthermore, the cyclin D related genes (CCND1, CCND2 and CCND3) were highly upregulated in keloids (FIG. 7b).

Expression of critical transcriptional regulators such as such as Activator protein 1 (AP1) transcription factors (JUN, JUNB, JUND, FOS, FOSB, FOSL1 and FOSL2) were significantly upregulated at day 1 and day 5; FBJ murine osteosarcoma viral oncogene homolog B (FOSB) and JunB Proto-Oncogene (JUNB) were also significantly upregulated at day 5 (FIG. 7b). However, cAMP response element binding protein (CREB)-related genes (CREB1, CREBBP and EP300) were not upregulated in keloid fibroblasts.

### Example 13

### Effects of Activin A in Human Dermal Fibroblasts from Normal and Keloid Tissues

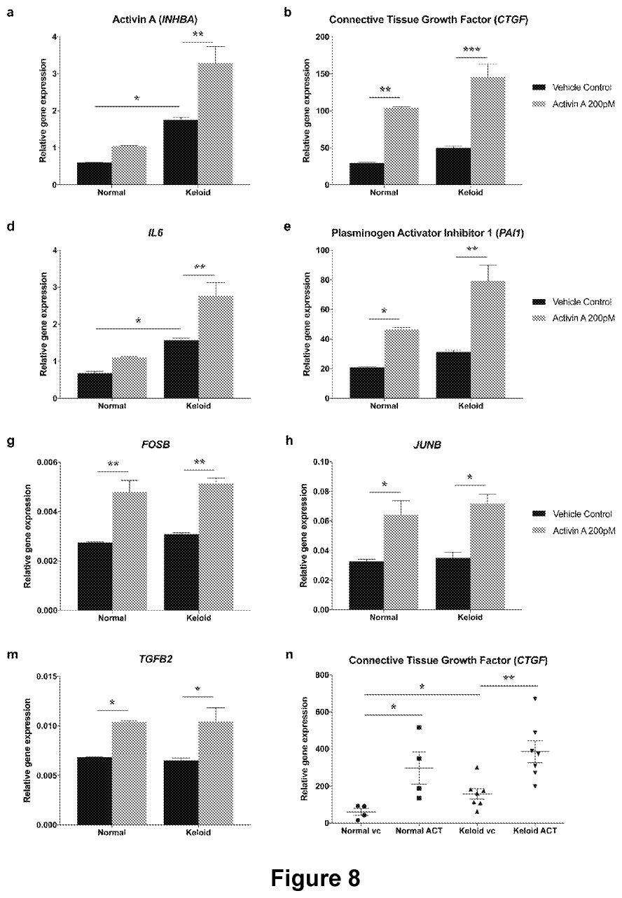

Treatment of normal and keloid fibroblast cultures from the same patient with 200 pM of exogenous activin A for 24 hours produced a further significant upregulation of activin A gene (INHBA) expression in the keloid fibroblasts (FIG. 8a). Exogenous treatment with Activin A also upregulated the expression of CTGF, IL6, PAH, FOSB, JUNB and TGFB2 in both normal and keloid fibroblasts (FIG. 8) from the same patient. The upregulation of CTGF by activin A was also confirmed in the multiple patient samples (FIG. 8n).

### Example 14

### Effects of Activator Protein 1 (AP1) Transcription Factor and Human Follistatin 288 (FST288) on Human Dermal Fibroblasts

To investigate the role of AP1 in keloid pathogenesis, fibroblasts were treated for 3 days with SR11302, an inhibitor of AP1 activity. At concentrations of 10 uM and 15 uM of SR11302, INHBA and CTGF gene expression significantly decreased in normal and keloids. However, normal and keloid fibroblasts in culture did not tolerate higher concentrations of SR11302 with doses higher than 20 uM resulting in cell death. Consistent with activin A gene expression, activin A protein in cell lysate and media were significantly decreased in both normal and keloid fibroblasts after treatment with AP1 inhibitor. Moreover, in order to examine the effects of FST288, fibroblasts were treated for 3 days or 5 days. At both day 3 and day 5, INHBA was significantly downregulated by the FST288 treatment in both normal and keloid fibroblasts.

Those skilled in the art will appreciate that the disclosure described herein is susceptible to variations and modifications other than those specifically described. It is to be understood that the disclosure contemplates all such variations and modifications. The disclosure also enables all of the steps, features, compositions and compounds referred to or indicated in this specification, individually or collectively, and any and all combinations of any two or more of the steps or features or compositions or compounds.

All patents, applications, publications, test methods, literature, and other materials cited herein are hereby incorporated by reference in their entirety as if physically present in this specification.

### BIBLIOGRAPHY

Amo et al. (2014) BUMS 40(7):1255-1266
Andrews et al. (2016) Matrix Biology 51:37-46
Ashcroft and Mills (2002) J Clin Invest 110(5):615-624
Gauglitz et al. (2011) Molecular Medicine 17(1-2):113-125
Gilliver et al. (2008) endocrinology 149(11):5747-5757
Guo et al. (2010) Journal Dent Res 89:219-229
Hedger et al. (2011) Vitam Horm 85:255-297
Hermiston et al. (2003) Annul Rev Immunol 21:107-137
Hubner et al. (1996) Dev Biol 173:490-498
Knight et al. (1996) J Endocrinol 148(2):267-279
Mukhopadhyay et al. (2007) Am J Physiol Cell Physiol 292:C1131-1338
Mung et al. (1999) EMBOJ 18:5205-5215
Mustoe et al. (2004) Amer Journal Surgery 187:655-705
O'Conner et al. (1999) Hum Reprod 14(3):827-832
Rapini et al. (2007) Dermatology: 2 volume set, St. Louis, Mosby at p 1499
Shih et al. (2010) Wound Repair Regeneration 18:139-153
Steed et al. (1997) 77(3):575-586
Usui et al. (2008) Journal of Histochem Cytochem 56:687-696

1. A clinical management protocol to assess likely extent of aberrant scar formation at the site of a wound or potential wound in a subject, said method comprising contacting a sample of fibroblasts from the healing area from the subject with an activin and screening for time-related sensitivity to the activin wherein a rapid change in gene, miRNA and/or protein expression profile, or other indicator of activin-mediated signaling, in response to activin compared to a control is indicative of a likelihood of aberrant scar development; wherein a slow change in expression profile compared to a control is indicative of a likelihood of non-aberrant scar development.

2. The protocol of claim 1 wherein the aberrant scar formation results from fibrosis or inflammation associated with a wound or skin condition.

3. The protocol of claim 1 or 2 wherein the subject is selected from the group consisting of a human, non-human primate, cow, sheep, horse, pig, goat, llama, alpaca, camel, dog, cat, mouse, rat, hamster, guinea pig and rabbit.

4. The protocol of claim 3 wherein the subject is a human.

5. The protocol of any one of claims 2 to 4 wherein the fibrosis or inflammation is associated with a condition selected from the group consisting of surgical trauma or injury, Dupuytren's disease, site of a microbial or viral infection, an insect bite, pimples or other skin lesions, area of psoriasis or scleroderma, eczema, a scratch mark, a stretch mark (striae), a hypertrophic scar, a burn, sunburn, a site of body piercing, a melanoma or cancer scar or dermatomyositis or other autoimmune disease.

6. The protocol of claim 5 wherein the Dupuytren's disease is Dupuytren's contracture.

7. The protocol of claim 5 wherein the skin lesion is an ulcer.

8. The protocol of any one of claims 2 to 7 wherein the fibrosis or associated inflammation is exacerbated by a condition selected from the group consisting of type 1 or 2 diabetes, obesity, aging, coronary heart disease, peripheral vascular disease, wound or skin infection, cancer including melanoma, immunosuppression and the effects of radiation or chemotherapy or site of catheterization or biopsy.

9. The protocol of any one of claims 1 to 8 wherein the wound is a skin wound.

10. The protocol of claim 9 wherein the skin wound affects one or more of the epidermal, dermal or hypodermal layers.

11. The protocol of claim 1 wherein the activin is activin A.

12. The protocol of claim 1 wherein the activin is activin B.

13. The protocol of claim 11 wherein the activin is activin AB.

14. The protocol of any one of claims 1 to 13 wherein a subject deemed likely to exhibit aberrant scarring or who does exhibit aberrant scarring is given an activin inhibitor or an inhibitor of a downstream signaling component.

15. The protocol of claim 14 wherein the downstream signaling component is connective tissue growth factor (CTGF).

16. The protocol of claim 14 or 15 wherein the activin inhibitor is a TGF-β antagonist, an AP-1 inhibitor, an inhibitor of cAMP response element binding (CREB) protein or an inhibitor of prostaglandin E2 (PGE2).

17. The protocol of claim 16 wherein the TGF-β antagonist is a TGF-β1, 2 or 3 antagonist.

18. The protocol of claim 16 or 17 wherein the TGF-β antagonist is follistatin, PB-01 or an AP-1 inhibitor or a functional variant or isoform thereof.

19. The protocol of any one of claims 14 to 18 further comprising the administration of an anti-androgen agent, an anti-microbial, an anti-viral agent, an antibiotic, insulin, an anesthetic or an estrogen.

20. The method of claim 19 wherein the anti-androgen is an anti-testosterone.

21. Use of an activin inhibitor in the manufacture of a medicament in the treatment or prevention of aberrant scar formation associated with a fibrotic condition or an inflammatory condition in or on a subject deemed at risk of aberrant scar formation based on the protocol of any one of claims 1 to 13.

22. An activin inhibitor for use in the topical treatment of a fibrotic condition or an inflammatory condition associated therewith in or on a subject deemed at risk of aberrant scar formation based on the protocol of any one of claims 1 to 13.

23. Use of claim 21 or the activin inhibitor of claim 22 wherein the fibrotic or inflammatory condition is of the skin.

24. Use of claim 21 or the activin inhibitor of claim 22 wherein the fibrotic or inflammatory condition is at an internal wound.

25. Use of claim 21 or the activin inhibitor of claim 22 wherein the internal wound is around the bowel or urinary tract.

26. Use of claim 21 or the activin inhibitor of claim 22 wherein the fibrotic condition is keloids.

27. Use of claim 21 or the activin inhibitor of claim 22 wherein the fibrotic condition is or is exacerbated by Dupuytren's disease, psoriasis, scleroderma, eczema, a hypertrophic scar, a burn, sunburn, melanoma or other cancer, site of catheterization or site of a biopsy.

28. Use of claim 21 or the activin inhibitor of claim 22 wherein the subject is a human.
